(19) United States
(12) Patent Application Publication
(10) Pub. No.: US 2025/0383331 A1
(43) Pub. Date: Dec. 18, 2025

(54) DOMICILE INDOOR AIR QUALITY ANALYSIS SYSTEM

(71) Applicant: State Farm Mutual Automobile Insurance Company, Bloomington, IL (US)

(72) Inventors: Dave Dohrmann, Mesa, AZ (US); Mike Janowiak, Bloomington, IL (US); Karthik Srinivasan, Peoria, AZ (US)

(73) Assignee: State Farm Mutual Automobile Insurance Company, Bloomington, IL (US)

(21) Appl. No.: 18/796,610

(22) Filed: Aug. 7, 2024

Related U.S. Application Data
(60) Provisional application No. 63/659,052, filed on Jun. 12, 2024.

(51) Int. Cl. G01N 33/00

(52) U.S. Cl. CPC G01N 33/0034; G01N 33/0063

(57) ABSTRACT

A system for analyzing an air quality of a domicile may (1) receive air quality metrics for one or more spaces of the domicile from one or more sensors communicatively coupled to the system; (2) analyzing, using a machine learning model, the air quality metrics for the one or more spaces of the domicile; and/or (3) detect, based upon the analysis of the air quality metrics, one or more anomalies within the air quality of the domicile. The system may (4) predict, using the machine learning model, a cause of the one or more anomalies; (5) generate, using the machine learning model, a recommendation to address the cause of the one or more anomalies; and/or (6) present, via a user interface, the recommendation to a user.

### FIELD OF THE INVENTION

The present disclosure relates generally to air quality systems. More particularly, some example embodiments relate to using an air quality system to analyze an indoor air quality of a domicile using an artificial intelligence (AI) model, such as a machine learning (ML) model.

### BACKGROUND

The indoor air quality of a space may affect the health and well-being of individuals occupying that space. For example, the indoor air quality of a residential building may impact the individuals who live in the residential building. It would be beneficial to provide an informed, data-based solution to assist residents in taking actions to improve the indoor air quality at their specific domicile (e.g., a home, an apartment building, a condominium, etc.).

### BRIEF SUMMARY

An air quality analysis system may be provided that detects anomalies within an air quality of a domicile based upon air quality metrics received from sensors within the domicile. For instance, the sensors may monitor levels of a particulate, of carbon dioxide (CO₂), etc. In some embodiments, a machine learning model may be trained to detect the anomalies within the air quality of the domicile based upon training data specific to the particular domicile (e.g., using data relating to and/or obtained from the specific domicile or using data from related domiciles, such as domiciles sharing one or more characteristics in common with the domicile). In this way, the air quality analysis system may offer recommendations and solutions according to equipment and devices within the particular domicile. Additionally, the recommendations and solutions may be generated to align with preferences and/or behavioral trends of one or more residents of the domicile.

In one aspect, a system for analyzing an air quality of a domicile may be provided. The system may include one or more local or remote processors, servers, transceivers, sensors, memory units, mobile devices, wearables, smart watches, smart contact lenses, smart glasses, augmented reality glasses, virtual reality headsets, mixed or extended reality glasses or headsets, voice bots, chatbots, ChatGPT bots, InstructGPT bots, Codex bots, Google Bard bots, and/or other electronic or electrical components, which may be in wired or wireless communication with one another. For example, in one instance, the system may include one or more processors and one or more non-transitory memories storing processor-executable instructions that, when executed by the one or more processors, cause the system to perform several operations, including (1) receiving air quality metrics for one or more spaces of the domicile from one or more sensors (such as one or more home-mounted, vehicle-mounted, mobile device, smart appliance, smart home, smart device, and/or other sensors); (2) analyzing, using a machine learning model, the air quality metrics for the one or more spaces of the domicile; and/or (3) detecting, based upon the analysis of the air quality metrics using the machine learning model, one or more anomalies within the air quality of the domicile. The computer system may include additional, less, or alternate functionality and/or operations, including that discussed elsewhere herein.

For instance, in certain embodiments, the one or more anomalies may include a monitored level of at least one of (a) volatile organic compounds (VOCs), (b) carbon monoxide (CO), (c) humidity, (d) temperature, (c) radon, (f) formaldehyde, (g) nitrogen dioxide (NO2), and/or (h) an air exchange rate exceeding a threshold level.

In some implementations, the functionality and/or operations may include assessing, using the machine learning model, an efficacy of an air quality improvement device using the air quality metrics, the air quality improvement device configured to improve the air quality of the domicile. The air quality improvement device may include at least one of an air filtration system or an air cleaning system. In certain embodiments, the functionality and/or operations may include (i) developing baseline air quality metrics for the domicile, and (ii) detecting the one or more anomalies based upon the baseline air quality metrics for the domicile.

Additionally or alternatively, the functionality and/or operations may include (i) predicting, by the machine learning model, a cause of the one or more anomalies; (ii) generating, by the machine learning model, one or more recommendations to address the cause of the one or more anomalies; and/or (iii) presenting, via a user interface, the one or more recommendations to a user (such as presenting a recommendation via text or graphics on a display, or audibly or verbally via chat bot or voice bot). In certain embodiments, the cause of the one or more anomalies may be determined based upon a cross-correlation between the air quality metrics and data from one or more devices in the domicile. Additionally or alternatively, the recommendation may include at least one of servicing a piece of equipment of the domicile or activating an air quality improvement device. The functionality and/or operations may also include automatically initiating the at least one of the servicing the piece of equipment of the domicile or activating the air quality improvement device in response to detecting the one or more anomalies.

In various implementations, the one or more sensors includes a plurality of sensors configured to be installed within a plurality of spaces of the domicile, the plurality of spaces including two or more of a bedroom, a family room, a living room, an office space, a kitchen, a bathroom, or a basement. In some implementations, (i) receiving the air quality metrics includes receiving the air quality metrics for the plurality of spaces; (ii) analyzing the air quality metrics includes analyzing, using the machine learning model, the air quality metrics for the plurality of spaces; and/or (iii) detecting the one or more anomalies includes detecting a first anomaly within the air quality of a first space of the plurality of spaces of the domicile. The functionality and/or operations may further include providing an indication of the first anomaly and the first space of the domicile for which the anomaly is detected.

In some implementations, the one or more sensors may include one or more indoor air quality sensors of the domicile and the air quality metrics may include indoor air quality metrics. The functionality and/or operations may include (i) receiving outdoor air quality metrics from at least one of one or more outdoor air quality sensors coupled to, or proximate to, an exterior of the domicile or an external system configured to provide outdoor air quality data for a geographic location of the domicile; and/or (ii) analyzing, using the machine learning model, the outdoor air quality metrics. In certain implementations, detecting the one or more anomalies may include detecting a first anomaly of the one or more anomalies by the machine learning model using both the indoor air quality metrics and the outdoor air quality metrics. In such implementations, the functionality and/or operations may include (i) determining, by the machine learning model, a covariance between a first indoor air quality metric of the indoor air quality metrics and a first outdoor air quality metric of the outdoor air quality metrics over a timeframe; and/or (ii) detecting the first anomaly responsive to the covariance of the first indoor air quality metric and the first outdoor air quality metric being less than a threshold covariance.

In another aspect, a computer-implemented method for analyzing an air quality of a domicile may be provided. The computer-implemented method may be implemented via one or more local or remote processors, servers, transceivers, sensors, memory units, mobile devices, wearables, smart watches, smart contact lenses, smart glasses, augmented reality glasses, virtual reality headsets, mixed or extended reality glasses or headsets, voice bots or chatbots, ChatGPT bots, InstructGPT bots, Codex bots, Google Bard bots, and/or other electronic or electrical components, which may be in wired or wireless communication with one another. In one instance, the computer-implemented method may include, such as via one or more local or remote processors, transceivers, sensors, other electronic components, including those discussed elsewhere herein, and/or computer-readable storage media having instructions stored thereon executable by the processors, transceivers, sensors, and/or other electronic components, (1) receiving air quality metrics for one or more spaces of the domicile from one or more sensors; (2) training machine learning model; (3) analyzing, using the machine learning model, the air quality metrics for the one or more spaces of the domicile; (4) detecting, based upon the analysis of the air quality metrics using the machine learning model, one or more anomalies within the air quality of the domicile; (5) predicting, using the machine learning model, a cause of the one or more anomalies, where the cause may be predicted using data from at least one of the one or more sensors or one or more other devices in the domicile; (6) generating, using the machine learning model, a recommendation to address the cause of the one or more anomalies; and/or (7) automatically initiating an action responsive to the recommendation. The method may include additional, less, or alternate functionality, including that discussed elsewhere herein.

For instance, the computer-implemented method may include, such as via one or more processors and/or other electronic components, training the machine learning model using domicile-specific training data. The domicile-specific training data may include training data specific to the domicile for which the computer-implemented method is analyzing the air quality. Additionally or alternatively, the computer-implemented method may include, such as via one or more processors and/or other electronic components, generating, using the machine learning model, a synthetic training dataset. In certain embodiments, the machine learning model is trained using the synthetic training dataset until the domicile-specific training data reaches a threshold amount.

In some implementations, the recommendation may include at least one of servicing a piece of equipment of the domicile or activating an air quality improvement device. In such implementations, the computer-implemented method may include, such as via one or more processors and/or other electronic components, presenting the recommendation to a user.

In certain embodiments, the one or more sensors may include one or more indoor air quality sensors of the domicile and the air quality metrics may include indoor air quality metrics. For instance, the computer-implemented method may include, such as via one or more processors and/or other electronic components, (i) receiving outdoor air quality metrics from at least one of one or more outdoor air quality sensors coupled to or proximate to an exterior of the domicile or an external system configured to provide outdoor air quality data for a geographic location of the domicile; (ii) determining, using the machine learning model, a covariance between a first indoor air quality metric of the indoor air quality metrics and a first outdoor air quality metric of the outdoor air quality metrics over a timeframe; and/or (iii) detecting the first anomaly responsive to the covariance of the first indoor air quality metric and the first outdoor air quality metric being less than a threshold covariance.

In another aspect, a non-transitory computer readable medium having instructions stored thereon that, when executed by one or more processors, cause the one or more processors to perform various functionality and operations. For instance, the functionality and operations may include or direct (1) developing, using a machine learning model, baseline air quality metrics for a plurality of spaces of a domicile; (2) receiving air quality metrics for the plurality of spaces of the domicile from a plurality of sensors configured to be installed within the plurality of spaces of the domicile; (3) analyzing, using the machine learning model, the air quality metrics for the plurality of spaces of the domicile based upon the baseline air quality metrics for the plurality of spaces of the domicile; (4) detecting, based upon the analysis of the air quality metrics using the machine learning model, a first anomaly within an air quality of a first space of the plurality of spaces of the domicile; (5) assessing, using the machine learning model, an efficacy of an air quality improvement device using the air quality metrics, the air quality improvement device configured to improve the air quality of the first space of the plurality of spaces of the domicile; (6) determining, based upon the efficacy of the air quality improvement device, a cause of the detected first anomaly; and/or (7) initiating a response to the first detected anomaly, where the response to the first detected anomaly includes a servicing of the air quality improvement device. The instructions may direct additional, less, or alternate functionality and/or operations, including that discussed elsewhere herein.

For instance, in certain embodiments, the air quality metrics may include at least one of (i) a particulate level or (ii) a carbon dioxide (CO₂) level.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers indicate identical, functionally similar, and/or structurally similar elements.

There are shown in the drawings arrangements which are presently discussed, it being understood, however, that the present embodiments are not limited to the precise arrangements and are instrumentalities shown, wherein:

FIG. 1 is a block diagram of an exemplary building management computer system, according to some embodiments.

FIG. 2 is a block diagram of an exemplary computer air quality analysis system, according to some embodiments.

FIG. 3 is a flow diagram of an exemplary computer-implemented or computer-based process for analyzing an air quality of a domicile, according to some embodiments.

FIG. 4 is a flow diagram of an exemplary computer-implemented or computer-based process of responding to one or more anomalies within an air quality of a domicile, according to some embodiments.

FIG. 5 is a depiction of an exemplary user interface including a recommendation to address a detected anomaly in an air quality of a domicile, according to some embodiments.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

Various example embodiments of the present disclosure relate to, inter alia, an indoor air quality (IAQ) analysis system for a domicile that detects, using machine learning, anomalies within air quality metrics. In some embodiments, the system detects the anomalies using one or more IAQ sensors in the domicile, such as to provide an analysis specific to the domicile in which the system is being implemented. For instance, different domiciles, and even different spaces within a same domicile, may have different characteristics that cause the domiciles/spaces to react differently to changes in IAQ-related parameters. For example, different domiciles/spaces may vary IAQ parameters differently to address particular needs and/or preferences of the residents at the domicile. Based upon the detected anomalies, the system may, using the machine learning, provide unique recommendations to a user of the system for alleviating the cause of the detected anomaly and improving the IAQ of the particular domicile.

### Overview

Referring to the Figures, computer systems and computer-implemented methods for analyzing an air quality of a domicile may be provided. For example, the computer system may be configured to receive air quality metrics (e.g., a particulate level, a carbon dioxide (CO₂) level, an amount of volatile organic compounds (VOCs), a carbon monoxide (CO) level, a humidity percentage, a temperature, a radon level, an amount of formaldehyde, a nitrogen dioxide (NO₂) level, an air exchange rate, etc.) for one or more spaces of the domicile (e.g., a bedroom, a living room, a family room, an office space, a basement, a kitchen, a bathroom, a patio, and any other space interior and/or exterior to the domicile) from one or more sensors coupled to the system (e.g., an indoor air quality sensor, an outdoor air quality sensor, and/or any other sensor configured to measure data associated with the domicile). The system may analyze the air quality metrics for the one or more spaces of the domicile using a machine learning model.

Based upon the analysis of the air quality metrics, the system may detect one or more anomalies (e.g., the particulate level, the carbon dioxide (CO₂) level, the amount of volatile organic compounds (VOCs), the carbon monoxide (CO) level, the humidity percentage, the temperature, the radon level, the amount of formaldehyde, the nitrogen dioxide (NO₂) level, and/or the air exchange rate failing to satisfy a threshold) within the air quality of the domicile. In some embodiments, a recommended response (e.g., activation of an air quality improvement device, servicing of a piece of equipment within the domicile, installation of an air quality improvement device, etc.) to the detected anomalies may be generated and provided via a user interface.

Advantageously, one aspect of the computer systems and computer-implemented methods described herein may allow individuals to analyze an air quality of their domicile and take action to maintain a healthy air quality. For example, by receiving air quality metrics for one or more spaces of the domicile from one or more sensors, analyzing the air quality metrics using a machine learning model, and detecting one or more anomalies within the air quality of the domicile based upon the analysis of the air quality metrics from the one or more sensors, the computer systems and computer-implemented methods described herein may, in certain embodiments, identify an air quality improvement recommendation according to the particular domicile that limits/reduces potential risks to a resident/occupant of the domicile (e.g., risk of living under unhealthy air quality conditions, risk of air quality improvement device failure, etc.). Accordingly, the systems and methods provide technological improvements to enhance health and comfort, reduce air quality risks, and streamline data analysis processes, thereby optimizing computational resources and system integrity.

One technical advantage of various embodiments of the present disclosure is optimization of air quality recommendations for a particular domicile by training a machine learning model to generate the recommendations using specific data relevant to the particular domicile and/or domiciles that are similar to the particular domicile. This technological improvement allows the system to select training data that may teach the machine learning model to generate the accurate analyses and relevant predictions for the air quality of the particular domicile. This technological advantage further improves processing power by reducing the amount of data that the system may use to perform the processes described herein. Another technical improvement provided by various implementations is reducing the amount of user (e.g., resident) intervention needed to detect air quality issues and perform the appropriate actions in response.

Further, example computer systems and computer-implemented methods described herein may be configured to provide individuals with protective services (e.g., coverage, etc.) over various air quality management systems, for example based upon an estimated efficacy of the associated air quality management systems, thereby providing individuals with increased coverage, reducing an individual's level of risk (e.g., injury or financial risk, etc.), and/or reducing an individual's resource consumption (e.g., financial resource consumption, etc.).

### Exemplary Building Management System with Air Quality Analysis System

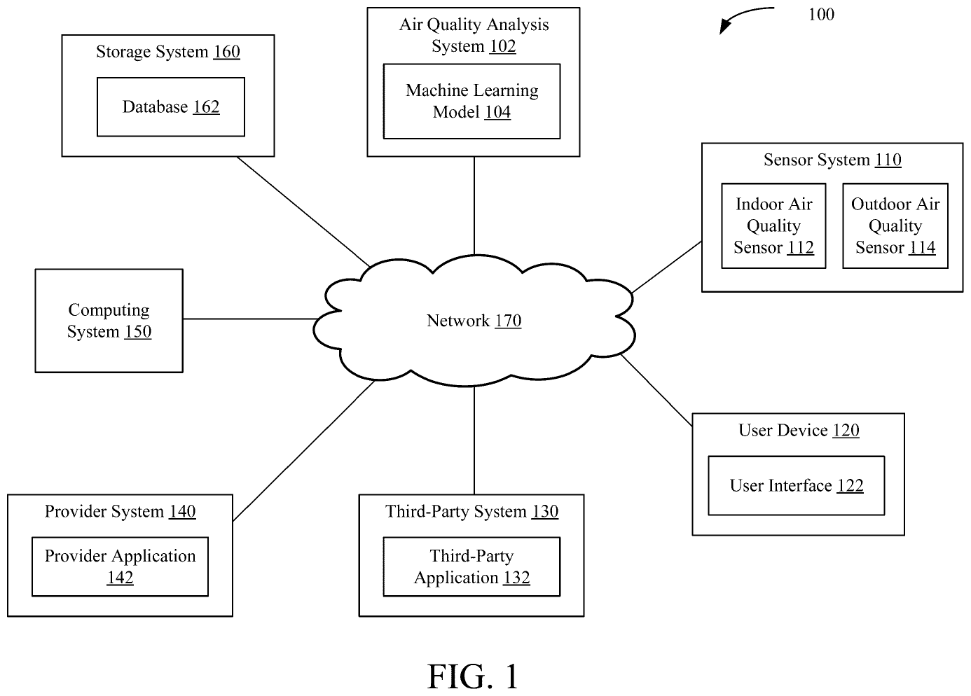

Referring to FIG. 1, a block diagram of an exemplary building management computer system, shown as building management system 100, is shown, according to some embodiments. The building management system 100 may include an air quality analysis computer system, shown as air quality analysis system 102 having a machine learning model 104, a sensor system 110 having at least one indoor air quality sensor 112 and at least one outdoor air quality sensor 114, and a user device 120 having a user interface 122. The building management system 100 may also include a third-party system 130 having a third-party application 132, a provider system 140 having a provider application 142, and a computing system 150. The building management system 100 may also include a storage system 160 having a database 162. The components of the building management system 100 may be connected, or in wired or wireless communication, via a network 170. It should be noted that the number and type of components shown are merely illustrative and, in various embodiments, implementations of the building management system 100 may have additional, fewer, and/or different components than those illustrated in FIG. 1, including those mentioned elsewhere herein.

Referring still to FIG. 1, according to some embodiments, the air quality analysis system 102 may be configured to communicate with components of the building management system 100. For example, information and/or data associated with the sensor system 110 and/or the user device 120 may be communicated to the air quality analysis system 102 (e.g., via the network 170). Information and/or data associated with the third-party system 130 and/or the provider system 140 may also be communicated to the air quality analysis system 102 (e.g., via the network 170). Information and/or data associated with the computing system 150 and/or the storage system 160 may also be communicated to the air quality analysis system 102 (e.g., via the network 170).

In some embodiments, the air quality analysis system 102 may be implemented using cloud computing services. The air quality analysis system 102 may be implemented using one or more computing devices, for example operating alone and/or in combination. The air quality analysis system 102 may be implemented using computing architectures like multiple distributed servers, and/or similar computing devices and/or systems. In various implementations, the air quality analysis system 102 may be another suitable computing system, for example distributed across multiple systems or devices (e.g., which may be located within a single building or facility, or distributed across multiple different buildings or facilities), or within a single computer (e.g., one server, housing, etc.). All such implementations are contemplated herein.

As shown in FIG. 1, the air quality analysis system 102 includes the machine learning model 104. The machine learning model 104 may utilize machine learning, generative artificial intelligence, or other advanced computing techniques. As such, the machine learning model 104 may employ supervised, unsupervised, and/or semi-supervised machine learning techniques, which may be followed by, and/or used in conjunction with, reinforced and/or reinforcement learning techniques. In some embodiments, the machine learning model 104 may generate audible or verbal output, text or textual output, visual or graphical output, output for use with speakers and/or display screens of a mobile computing device, and/or other types of output for user and/or other computer consumption.

Noted above, the machine learning model 104 may be configured to implement machine learning, such that the machine learning model 104 “learns” to analyze, organize, and/or process data without being explicitly programmed. For example, as described herein, the machine learning model 104 may be configured to receive air quality related information (e.g., air quality metrics from the sensor system 110, historic air quality related information, domicile-specific training data, etc.) such that the machine learning model 104 is trained to analyze air quality metrics for a particular domicile and generate recommendations related to the air quality of the particular domicile. In certain embodiments, as described herein, the machine learning model 104 may be trained to generate an air quality index for the particular domicile. As the air quality analysis system 102 receives additional data (e.g., by performing the functions described herein over time), the machine learning model 104 is retrained based upon the additional data such that the machine learning model 104 may perform more accurate analyses and generate more relevant recommendations for the particular domicile in which the air quality analysis system 102 is analyzing the air quality.

As shown, information/data associated with the sensor system 110 may be communicated to the air quality analysis system 102. The sensor system 110 may be configured to communicate information/data to the machine learning model 104. In some embodiments, a device coupled to a system or device monitoring an air quality metric, a device obtaining data from and/or regarding an air quality metric, and/or another suitable system or device associated with an air quality metric may be configured to communicate information/data to the air quality analysis system 102. For example, the air quality metric may include a particulate level, a carbon dioxide (CO₂) level, etc.

Each component of the sensor system 110 (e.g., the indoor air quality sensor 112, the outdoor air quality sensor 114, etc.) may be associated with a space within a domicile. The domicile refers to a place of residence for a user (e.g., a user or operator associated with the user device 120, a customer of the provider associated with the provider system 140, a resident of the domicile, etc.). For instance, the space may be controlled and/or occupied by one or more users (e.g., the user or operator associated with the user device 120, the customer of the provider associated with the provider system 140, the resident of the domicile, etc.). The space may be any kind of space, including a bedroom, a family room, a living room, an office space, a basement, a kitchen, a bathroom, a hallway, a garage, a patio, a backyard, a front yard, a porch, a garden, and/or any other space associated with the domicile.

The sensor system 110 may include at least one indoor air quality sensor (e.g., also referred to herein as indoor air quality sensor 112). The indoor air quality sensor 112 may include a device configured to measure one or more indoor air quality metrics for a space that is internal to the domicile. In certain embodiments, the indoor air quality sensor 112 may be positioned (e.g., installed, etc.) within the domicile such that the space for which the indoor air quality sensor 112 measures the one or more air quality metrics includes space inside the domicile (e.g., the bedroom, the family room, the living room, the office space, the kitchen, the bathroom, the basement, the hallway, any other space internal to the domicile, etc.).

In some embodiments, the sensor system 110 may include at least one outdoor air quality sensor (e.g., also referred to herein as outdoor air quality sensor 114). The outdoor air quality sensor 114 may include a device configured to measure one or more outdoor air quality metrics for a space that is external to a domicile (e.g., the patio, the backyard, the front yard, the porch, the garden, any other space external to the domicile, etc.). For instance, the outdoor air quality sensor 114 may refer to an external system (e.g., external to the domicile, etc.) configured to provide outdoor air quality metrics for a geographic location of the domicile. That is, the outdoor air quality sensor 114 may be configured to measure an air quality (e.g., air pollution, etc.) specific to the geographic location of the domicile.

In various embodiments, the sensor system 110 may further include any other device (e.g., an Internet of Things (IoT) device, etc.), installed within and/or exterior to the domicile. For instance, the sensor system 110 may include a device configured to communicate information/data that is not related to an air quality metric (e.g., visual data, audio data, electricity data, security information, etc.) to the air quality analysis system 102.

The air quality analysis system 102 may be configured to receive historic air quality related information associated with the sensor system 110. More specifically, the machine learning model 104 may be configured to receive the historic air quality related information associated with the sensor system 110. In this way, the machine learning model 104 may be trained using the historic air quality related information and may generate recommendations related to an air quality analysis using the historic air quality related information. For example, the air quality analysis system 102 (e.g., the machine learning model 104) may receive information relating to historic temperature measurements, historic air exchange rates, historic levels of humidity, volatile organic compounds (VOCs), carbon monoxide (CO), radon, formaldehyde, nitrogen dioxide (NO₂), and/or other suitable air quality related information associated with the sensor system 110.

In various implementations, the air quality analysis system 102 may receive the historical air quality related information associated with a domicile in which the air quality analysis system 102 is analyzing the air quality and/or associated with other domiciles that are related to the domicile in which the air quality analysis system 102 is analyzing the air quality. For example, if the domicile in which the air quality analysis system 102 is analyzing the air quality is a one bedroom apartment in a complex, the historic air quality related information may include historic air quality related information associated with a sensor system 110 in other one bedroom apartments in other complexes. Additionally or alternatively, the historic air quality related information may include data from sensors that are not within, on, or proximate to a domicile. For example, the historic air quality related information may include historic information from a weather service regarding a temperature, a humidity, an air quality, etc., for a geographical region related to the domicile in which the air quality analysis system 102 is analyzing the air quality.

As shown, the air quality analysis system 102 may be configured to communicate with the user device 120. The user device 120 may include one or more human-machine interfaces or client interfaces, shown as user interface 122 (e.g., a graphical user interface, a text-based computer interface, a client-facing web service, a web service that provides pages to a web client, etc.), for example for controlling, viewing, and/or otherwise interfacing with the air quality analysis system 102. The user device 120 may include a personal mobile computing device (e.g., a smart phone, a tablet, a mobile device, a wearable, smart glasses, a smart watch, etc.). The user device 120 may include a computer workstation, a client terminal, a remote or local interface, and/or any other user interface device. The user device 120 may be a stationary terminal (e.g., a desktop computer, a laptop computer, a tablet, or another suitable non-mobile device).

In some implementations, information/data associated with the user device 120 may be communicated to the air quality analysis system 102. In certain embodiments, the user device 120 itself may be configured to communicate information/data to the air quality analysis system 102. In some implementations, a device coupled to the user device 120, a component implemented with the user device 120, an application or program housed and/or executed on the user device 120, and/or another suitable component associated with the user device 120 may be configured to communicate information/data to the air quality analysis system 102. The information/data associated with the user device 120 may be communicated to the machine learning model 104 such that the machine learning model 104 may be trained using the information/data associated with the user device 120.

The air quality analysis system 102 may also be configured to receive information/data associated with a user or operator associated with the user device 120. For example, the user device 120 may (e.g., automatically, or in response to an input from a user or operator, etc.) be configured to communicate information associated with a user or operator associated with one or more applications (e.g., housed or executed on the user device 120). In some embodiments, the user device 120 may also be configured to communicate information associated with trends or tendencies of a user or operator. The air quality analysis system 102 may also be configured to receive information associated with a product or service associated with a user or operator of the user device 120. According to some embodiments, the user device 120 may be configured to communicate historic information/data associated with a user or operator to the air quality analysis system 102, as well as information in real-time. The information/data associated with the user or operator may be communicated to the machine learning model 104 such that the machine learning model 104 may be trained using the information/data associated with the user or operator of the user device 120.

The air quality analysis system 102 may also be configured to receive data or information gathered and/or captured by the user device 120. For example, the user device 120 may include a microphone or camera (e.g., for capturing audiovisual information). The user device 120 may capture (e.g., automatically, and/or in response to an input by a user or operator) audiovisual data around the user device 120, for example while a user or operator is in a domicile. The user device 120 may communicate the audiovisual information to the air quality analysis system 102. In some embodiments, the user device 120 may be configured to communicate audiovisual information (e.g., voice memos, voicemails, images, videos, etc.) stored on the user device 120 to the air quality analysis system 102.

As shown, the air quality analysis system 102 may be configured to receive information/data associated with the third-party system 130. The third-party system 130 may include a third-party application 132. While the building management system 100 is shown to include one third-party system 130, it is contemplated herein that the building management system 100 may include a plurality of third-party systems 130. The air quality analysis system 102 may be configured to receive air quality related information/data associated with the third-party system 130. For example, the third-party system 130 may include a weather service. The weather service may be configured to provide weather related information (e.g., a temperature, an air quality, a humidity, etc.) corresponding to a geographic region associated with a domicile in which the air quality analysis system 102 is analyzing the air quality. In certain embodiments, the weather service may be configured to provide historic weather-related information and/or weather related information in real time. The information/data associated with the third-party system 130 may be communicated to the machine learning model 104 such that the machine learning model 104 may be trained using the information/data associated with the third-party system 130.

As shown, information/data associated with the provider system 140 may be communicated to the air quality analysis system 102. The provider system 140 may be configured to communicate information/data to the air quality analysis system 102. In some embodiments, a device coupled to, a component implemented within the provider system 140, an application or program housed and/or executed on the provider system 140, and/or another suitable component associated with the provider system 140 may be configured to communicate information/data to the air quality analysis system 102.

The provider system 140 may include a provider application 142. In certain embodiments, the provider system 140 may be associated with a company or entity that provides protective services (e.g., insurance, etc.) to a user or operator (e.g., a user or operator associated with the user device 120), a company or service provider (e.g., OEM or a provider associated with the third-party system 130), and/or over one or more products or services (e.g., associated with the sensor system 110, etc.). In certain embodiments, the provider system 140 may include the air quality analysis system 102, as described herein. The provider system 140 may be configured to communicate with the air quality analysis system 102 (and/or the user device 120), for example to provide one or more air quality improvement recommendations and/or policy parameters. In some embodiments, the information/data associated with the provider 140 may be communicated to the machine learning model 140 such that the machine learning model 104 may be trained using the information/data associated with the provider system 140.

In various embodiments, the air quality analysis system 102 may be configured to receive an insurance policy parameter. The provider system 140 may be configured to provide a policy parameter (e.g., to the air quality analysis system 102, to the user device 120, to other components of the building management system 100, etc.). A policy parameter may refer to a parameter of one or more insurance products (e.g., coverages, policy terms/limits, premiums, etc.).

The policy parameter may be selected, generated, and/or offered, for example to supplement or increase existing coverage or to provide new coverage. In some embodiments, the provider system 140 may be configured to generate a plurality of policy parameters. For example, the provider system 140 may be configured to generate a plurality of policy parameters associated with one or more recommended air quality responses, as will be described herein.

In various embodiments, the policy parameters may be selected, generated, and/or offered based upon a policy availability and/or policy source, a policy availability location, and/or additional parameters (e.g., a cost, a time over which the policy is available, a product or service over which the policy is available, a destination range or location over which the policy is available, eligibility requirements, an ability to group or bundle different policies or parameters, available discounts or rewards associated with a policy or parameter, etc.).

As noted herein, the air quality analysis system 102 may be configured to receive one or more policy parameters associated with one or more recommended air quality responses. For example, a policy parameter may be generated (e.g., via the provider system 140) that provides coverage over a domicile equipped with one or more air quality improvement devices. The machine learning model 104 may use the one or more policy parameters associated with one or more recommended air quality responses to generate recommendations that allow for a domicile to receive coverage provided by the policy parameter.

Additionally or alternatively, the one or more policy parameters for a domicile may be generated using a plurality of parameters or factors. For example, a policy parameter (e.g., a policy cost or premium, etc.) may be generated for a domicile based upon a base policy (e.g., cost, rate, coverage, etc.), a location rating factor (e.g., city, state, urban location, rural location, etc.), a coverage rating (e.g., availability, amount, term, etc. of coverage), a claim rating factor (e.g., based upon historical claim information associated with a domicile and/or a user or operator, etc.), a discount or other cost saving, and/or a combination thereof. The one or more policy parameters may be selected and/or generated, for example to provide a premium discount and/or expanded coverage to domiciles that are associated with air quality metrics that indicate a suitable air quality for habitation in the domicile. The machine learning model 104 may be configured to use the one or more policy parameters to generate recommendations associated with an air quality of a domicile.

As shown, the air quality analysis system 102 may be configured to communicate with the computing system 150. In some embodiments, the computing system 150 may be a cloud-based computing system, for example to provide digital connections between different computing devices and/or systems (e.g., as described herein). The computing system 150 may be a virtual reality (VR) system or augmented reality (AR) system, for example to provide digital connections between a plurality of metadata sources, where the metadata sources are integrated within the VR system or AR system.

In various embodiments, the computing system 150 may be implemented using one or more computing devices, for example operating alone and/or in combination. In some embodiments, the computing system 150 may be implemented using computing architectures like multiple distributed servers, and/or similar computing devices and/or systems. In certain embodiments, the computing system 150 may be a server (e.g., including a processor coupled to a memory), for example to store and/or recall data and applications within the memory. In various other embodiments, the computing system 150 may be another suitable computing system, for example distributed across multiple systems or devices (e.g., which may be located within a single building or facility, or distributed across multiple different buildings or facilities), or within a single computer (e.g., one server, housing, etc.). All such implementations are contemplated herein.

As shown, the air quality analysis system 102 may be configured to communicate with the storage system 160 (e.g., having the database 162). In some embodiments, the air quality analysis system 102 communicates with the storage system 160, either directly (e.g., via the network 170) or indirectly (e.g., via the sensor system 110, the user device 120, etc.). The storage system 160 may include one or more devices (e.g., RAM, ROM, Flash memory, hard disk storage, etc.) for storing data and/or computer code for implementing and/or facilitating the various processes, layers, and/or circuits described herein. The storage system 160 may be or include volatile memory or non-volatile memory, and may include database components, object code components, script components, and/or any other type of information structure for supporting the various activities and information structures described herein.

In certain embodiments, and as will be discussed in greater detail, the air quality analysis system 102 may also be configured to generate data. For example, the air quality analysis system 102 may include components (e.g., a data compiler, an air quality metric analyzer, an anomaly detector, a recommendation generator, and a database) that obtain, analyze, process, generate, store, and/or communicate data. The components of the air quality analysis system 102 may perform the processes described herein wholly or at least partially using the machine learning model 104.

### Exemplary Air Quality Analysis Computer System

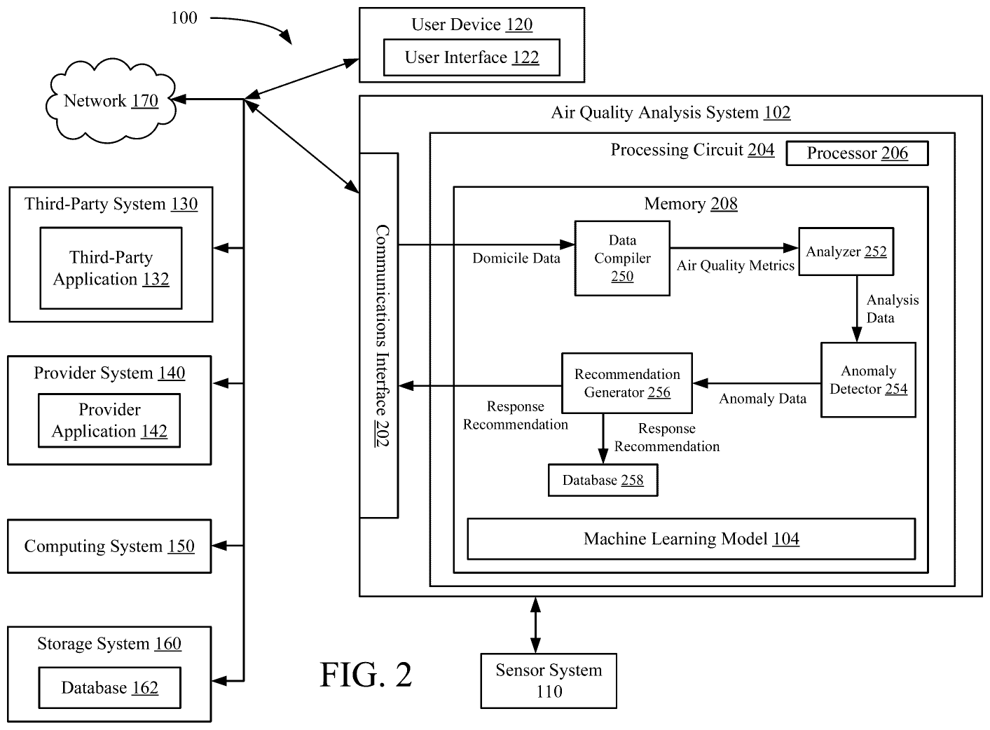

Referring now to FIG. 2, a block diagram of the exemplary air quality analysis system, e.g., the air quality analysis system 102, is shown in greater detail, according to some embodiments. As shown in FIG. 2, the air quality analysis system 102 may be communicably connected to the sensor system 110 (e.g., the indoor air quality sensor 112, the outdoor air quality sensor 114, etc.), the user device 120, the third-party system 130, the provider system 140, the computing system 150, and the storage system 160 (e.g., via the network 170). The air quality analysis system 102 may be communicably connected to other suitable systems and/or devices (e.g., via the network 170), including those devices mentioned elsewhere herein. It should be understood that some or all of the components of the air quality analysis system 102, the sensor system 110, the user device 120, the third-party system 130, the provider system 140, the computing system 150, the storage system 160, and/or the network 170 may be implemented as part of a cloud-based computing system configured to obtain, process, and/or communicate data from one or more external devices or sources.

Similarly, some, or all, of the components of the air quality analysis system 102, the sensor system 110, the user device 120, the third-party system 130, the provider system 140, the computing system 150, the storage system 160, and/or the network 170 may be integrated within a single device or be distributed across multiple separate systems or devices. In certain embodiments, the air quality analysis system 102, the sensor system 110, the user device 120, the third-party system 130, the provider system 140, the computing system 150, the storage system 160, and/or the network 170 are components of a controller, a device controller, a field controller, a computer work station, a client device, and/or another system or device that receives, processes, and/or communicates data from/to devices or other data sources.

As shown, the air quality analysis system 102 may include a communications interface 202, a processing circuit 204 having one or more processors 206, and one or more memories 208 (e.g., one or more computer-readable storage media having instructions stored thereon that are executed by the one or more processors 206) having the machine learning model 104. While the processing circuit 204, the processor 206, and the memory 208 are described herein in the singular for brevity, it should be understood that various embodiments may utilize two or more processing circuits, processors, and/or memories/computer-readable storage media in combination to perform the functions described herein with the singular components, and all such embodiments are contemplated within the scope of the present disclosure.

The communications interface 202 may include wired or wireless communications interfaces (e.g., jacks, antennas, transmitters, receivers, transceivers, wire terminals, etc.) for communicating data between the air quality analysis system 102 and external systems or devices (e.g., the sensor system 110, the user device 120, the third-party system 130, the provider system 140, the computing system 150, the storage system 160, etc.). In some embodiments, the communications interface 202 facilitates communications between the air quality analysis system 102 and one or more external applications and/or interfaces (e.g., the user interface 122, the third-party application 132, the provider application 142 etc.), for example to allow a remote user or operator to control, monitor, and/or adjust components of the air quality analysis system 102.

Further, the communications interface 202 may be configured to communicate with external systems and/or devices using any of a variety of communications protocols (e.g., HTTP(S), WebSocket, CoAP, MQTT, etc.) and/or any of a variety of other protocols. Advantageously, the air quality analysis system 102 may obtain, ingest, and process data from any type of system or device, regardless of the communications protocol used by the system or device.

As shown, the air quality analysis system 102 may include the processing circuit 204 having the processor 206 and the memory 208. While shown as single components, it should be appreciated that the air quality analysis system 102 may include one or more processing circuits, including one or more processors and memory.

In some embodiments, the air quality analysis system 102 may include a plurality of processors, memories, interfaces, and/or other components distributed across multiple devices or systems, which are communicably coupled via a network (e.g., the network 170). For example, in a cloud-based or distributed implementation, the air quality analysis system 102 may include multiple discrete computing devices, each of which includes a processor 206, memory 208, communications interface 202, and/or other components of the air quality analysis system 102. Tasks performed by the air quality analysis system 102 may be distributed across multiple systems or devices, which may be located within a single building or facility or distributed across multiple buildings or facilities. In other embodiments, the air quality analysis system 102 itself may be implemented within a single computer (e.g., one server, one housing, etc.). All such implementations are contemplated herein.

The processor 206 may be a general purpose or specific purpose processor, an application specific integrated circuit (ASIC), one or more field programmable gate arrays (FPGAs), a group of processing components, or other suitable processing components. The processor 206 may further be configured to execute computer code or instructions stored in the memory 208 or received from other computer readable media (e.g., USB or other local storage, network storage, a remote server, etc.).

The memory 208 may include one or more devices (e.g., memory units, memory devices, storage devices, etc.) for storing data and/or computer code for completing and/or facilitating the various processes described in the present disclosure. For example, as shown in FIG. 2, the memory 208 may include the machine learning model 104, and the machine learning model 104 may be configured to complete and/or facilitate the various processes described in the present disclosure. The memory 208 may include random access memory (RAM), read-only memory (ROM), hard drive storage, temporary storage, non-volatile memory, flash memory, optical memory, or any other suitable memory for storing software objects and/or computer instructions. In some implementations, the memory 208 may include database components, object code components, script components, and/or any other type of information structure for supporting the various activities and information structures described in the present disclosure. The memory 208 may be communicably connected to the processor 206 via the processing circuit 204, and may include computer code for executing (e.g., by the processor 206) one or more processes described herein. When the processor 206 executes instructions stored in the memory 208, the processor 206 may configure the processing circuit 204 to complete such activities.

As shown, the air quality analysis system 102 (e.g., the memory 208) may include a data compiler 250, an air quality metrics analyzer, shown as an analyzer 252, an anomaly detector 254, a recommendation generator 256, and a database 258. The following paragraphs describe some of the general functions performed by each of the components 250-258 of the air quality analysis system 102. In some embodiments, the machine learning model 104 may wholly or partially perform the functions performed by each of the components 250-258, as described herein. It should be noted that the number and type of components shown are merely illustrative and, according to various embodiments, implementations of the air quality analysis system 102 may have additional, fewer, and/or different components than those illustrated in FIG. 2.

The data compiler 250 may be configured to obtain input data, analyze the input data, and/or generate output data to be communicated to other components of the air quality analysis system 102. For example, the data compiler 250 may obtain (e.g., receive, request, pull, etc.) domicile data. The domicile data includes data/information relating to the domicile in which the air quality analysis system 102 is analyzing an air quality. The domicile data may be received from an external system or device (e.g., an edge device, the sensor system 110, the user device 120, etc.), for example via the communications interface 202. In some embodiments, the data compiler 250 may obtain the input data, analyze the input data, and/or generate the output data to be communicated to other components of the air quality analysis system 102 using the machine learning model 104.

According to some embodiments, the domicile data may include information associated with a sensor (e.g., the indoor air quality sensor 112, the outdoor air quality sensor 114, etc.). In certain implementations, the indoor air quality sensor 112 and/or the outdoor air quality sensor 114 is one of a plurality of sensors, such that the domicile data may include information associated with a plurality of sensors. For example, the domicile data may include domicile data from each of the indoor air quality sensor 112 and/or the outdoor air quality sensor 114. The domicile data may also include historic sensor information of each of the sensors. In various embodiments, the domicile data may include information associated with a user device (e.g., the user device 120). Additionally or alternatively, the domicile data may include information associated with a user or operator of a user device (e.g., the user device 120 or other computing device(s), including those mentioned herein).

In certain embodiments, the domicile data may include information associated with a product or service associated with a user or operator of a user device (e.g., the user device 120 or other computing device(s), including those discussed herein.). In certain embodiments, the domicile data may include information collected and/or gathered via a user device (e.g., the user device 120). For example, the domicile data may include audiovisual information (e.g., captured via a microphone or camera of the user device 120, and/or captured via a mobile device, AR glasses, VR headset, voice bot, chatbot, wearable, or other computing devices, including those mentioned herein), for example audiovisual (including audio, visual, video, image, and/or graphical information, data, and sensor data) information and related data captured in real-time and/or historical audiovisual information and related data.

The domicile data may further include information associated with a third-party system (e.g., the third-party system 130). In some embodiments, the third-party system 130 is one of a plurality of third-party systems 130, such that the domicile data may include information associated with a plurality of third-party systems 130.

In various embodiments, the domicile data may include information/data (e.g., received from the third-party system 130) relating to other domiciles that share one or more common characteristics with the domicile in which the air quality analysis system 102 is analyzing the air quality. The one or more common characteristics may include characteristics of the domicile (e.g., a one bedroom apartment in a complex, a two bedroom detached condo, a four bedroom single family home, a 2,000 square foot ranch-style home, etc.), characteristics of one or more spaces within the domicile (e.g., a 100 square foot bedroom, a 300 square foot kitchen, an unfinished basement, a half bath, etc.), characteristics of one or more residents of the domicile (e.g., a married couple, an immune-compromised child, a retired adult, a five-person family, etc.) characteristics of one or more owners of the domicile (e.g., a commercial entity, a single adult, a married couple, a landlord, etc.). For example, the characteristics of the one or more residents of the domicile may be provided using behavioral data, preference data, demographic data, etc. In some such embodiments, the machine learning model 104 may be trained initially as a general model relating to domiciles with similar characteristics, and then may be retrained over time using data from the particular domicile such that the recommendations are more specific to that domicile and/or the residents of that domicile.

In certain embodiments, the domicile data may include information associated with a provider system 140. The provider system 140 may be associated with a company that provides protective services (e.g., insurance, etc.) to a user or operator, a company, a service provider, and/or one or more products or services. The domicile data may include one or more policy parameters associated with one or more users, operators, companies, service providers, products, and/or services. The domicile data (e.g., one or more policy parameters) may be provided using historical policy parameter information (e.g., historic policy characteristics, etc.), and/or one or more additional policy parameters (e.g., cost, discounts, availability and/or policy source, a policy availability location, a time over which the policy is available, a product or service over which the policy is available, a destination range or location over which the policy is available, eligibility requirements, ability to group or bundle different policies or parameters, available discounts or rewards associated with a policy or parameter, etc.), as described herein.

In some embodiments, the domicile data may include information associated with a computing system (e.g., the computing system 150) and/or a storage system (e.g., the storage system 160). The domicile data may include historic and/or real-time air quality information, for example from (e.g., directly, or indirectly) the computing system 150 and/or the storage system 160, as described herein. In certain implementations, the domicile data may be received by the air quality analysis system 102 in real-time and/or at one or more series or intervals (e.g., hourly, daily, etc., automatically in response to an air quality event initiated and/or associated with the sensor system 110, user device 120, the third-party system 130, the provider system 140, etc.).

As shown, the data compiler 250 may be configured to obtain input data (e.g., the domicile etc.), analyze the input data, and/or generate output data. For example, the data compiler 250 (e.g., using the machine learning model 104) may be configured to obtain (e.g., receive, request, pull, etc.) domicile data, analyze (e.g., compile, process, etc.) the data, and generate air quality metrics. The air quality metrics may be communicated to another component of the air quality analysis system 102 (e.g., the analyzer 252). In certain embodiments, the air quality metrics may include data associated with the sensor data.

The analyzer 252 may be configured to obtain input data, analyze the input data, and/or generate output data to be communicated to other components of the air quality analysis system 102. In some embodiments, the analyzer 252 may obtain the input data, analyze the input data, and/or generate the output data to be communicated to other components of the air quality analysis system 102 using the machine learning model 104. For example, the analyzer 252 (e.g., the machine learning model 104) may obtain (e.g., receive, request, pull, etc.) air quality metrics, analyze the air quality metrics, and/or generate analysis data, for example including a plurality of scores (e.g., associated with each of the air quality metrics) associated with the air quality of the domicile. As described herein, the analysis data may include a score for each of the plurality of air quality metrics. In this regard, the analyzer 252 (e.g., using the machine learning model 104) may be configured to generate analysis data that indicates an air quality metric does not satisfy a threshold score (e.g., amount).

According to certain embodiments, the analysis data generated by the analyzer 252 may include an air quality index for the domicile. The analyzer 252 may be configured to generate the air quality index using a plurality of factors, such as the domicile data received by the data compiler 250. For example, the analyzer 252 may generate the air quality index using a weighted combination of each of the plurality of factors (e.g., the domicile data) to generate a single score (e.g., a numerical score, a categorical score, a textual score, etc.) that represents a relative level of the air quality in the domicile. In some embodiments, the analyzer 252 may use a weighted combination of each score for each of the plurality of air quality metrics, as described above, to generate the single score. In this way, the single score may be more heavily influenced by the air quality metrics that may have a greater impact on the air quality of a particular domicile than the air quality metrics that may have less of an impact on the air quality of the particular domicile. The indoor air quality index may function to provide an easily understood, quick indication of the relative air quality of the domicile in a similar fashion to how an outdoor or ambient air quality index provides an indication of the relative quality of the outside air in an area.

In one example embodiment, the air quality index may be generated based on a weighted combination of volatile organic compounds (VOCs), carbon monoxide (CO) level, humidity percentage, and air exchange rate, where each of these factors could be weighted by a particular weight that may be the same or different as the other weights. In some implementations, individual scores for the component factors may be generated (e.g., normalized scores on a scale), and the individual scores may be combined in a weighted combination to generate the overall indoor air quality index for the domicile. In some implementations, the overall air quality index and/or the individual component scores may be provided to a user and/or used to implement one or more other actions, such as automated actions to improve the air quality index.

In various embodiments, the analyzer 252 may be configured to generate the analysis data using information associated with a sensor (e.g., the indoor air quality sensor 112 and/or the outdoor air quality sensor 114). In certain implementations, the analyzer 252 may be configured to generate the analysis data using information associated with a user device (e.g., the user device 120). The analyzer 252 may generate the analysis data using information associated with a user or operator of the user device 120 and/or a product or service associated with a user or operator of the user device 120, as described herein.

The analyzer 252 may be configured to generate the analysis data using information associated with a third-party system (e.g., the third-party system 130). In some embodiments, the analyzer 252 may be configured to generate the analysis data using information associated with a provider system (e.g., the provider system 140). For example, the analyzer 252 may be configured to generate the analysis data using a policy parameter (e.g., associated with one or more of a user, operator, company, service provider, and/or one or more products or services). In certain embodiments, the analyzer 252 may generate the analysis data using historical policy parameter information (e.g., historic policy characteristics, etc.), and/or one or more additional policy parameters (e.g., a cost associated with a policy, availability and/or policy source, a time over which the policy is available, coverage associated with a policy, etc.), as described herein.

Additionally or alternatively, the analyzer 252 (e.g., the machine learning model 104) may be configured to generate the analysis data using information associated with a computing system (e.g., the computing system 150) and/or a storage system (e.g., the storage system 160). For example, the analyzer 252 (e.g., the machine learning model 104) may generate the analysis data using historic and/or real-time air quality data associated with the storage system 160 and/or the computing system 150.

The anomaly detector 254 may be configured to obtain input data, analyze the input data, and/or generate output data to be communicated to other components of the air quality analysis system 102. In some embodiments, the anomaly detector 254 may obtain the input data, analyze the input data, and/or generate the output data to be communicated to other components of the air quality analysis system 102 using the machine learning model 104. For example, the anomaly detector 254 (e.g., the machine learning model 104) may obtain (e.g., receive, request, pull, etc.) air quality metrics, analyze the air quality metrics, and/or generate anomaly data. For example, the anomaly detector 254 (e.g., the machine learning model 104) may receive monitored levels (e.g., by the sensor system 110) of at least one of volatile organic compounds (VOCs), carbon monoxide (CO), humidity, temperature, radon, formaldehyde, nitrogen dioxide (NO₂), or an air exchange rate from the analyzer 252. The anomaly detector 254 may also receive, from the analyzer 252, an indication that at least one of the monitored levels does not satisfy (e.g., exceeds, falls short of, etc.) a threshold amount. For example, the threshold amount may include a standard defined by a health agency or regulatory entity. The threshold amount may differ between domiciles depending on the residents of and other parameters associated with each particular domicile.

For example, a first policy parameter associated with a first domicile may require that an air exchange rate reach a first threshold amount, while a second policy parameter associated with a second domicile may require that the air exchange rate reach a second threshold amount that is less than the first threshold amount. Therefore, the anomaly detector 254 may be configured to detect an anomaly within the air quality of the first domicile, but may not detect an anomaly within the air quality of the second domicile.

The recommendation generator 256 may be configured to obtain input data, analyze the input data, and/or generate output data to be communicated to other components of the air quality analysis system 102. The recommendation generator 256 may obtain the input data, analyze the input data, and/or generate the output data to be communicated to other components of the air quality analysis system 102 using the machine learning model 104. For example, the recommendation generator 256 (e.g., the machine learning model 104) may be configured to obtain (e.g., receive, request, pull, etc.) anomaly data, analyze the anomaly data, and generate an air quality improvement recommendation.

For example, the recommendation generator 256 (e.g., the machine learning model 104) may generate an air quality improvement recommendation that includes a user interface (or voice bot or chat bot, such as a ChatGPT bot) that provides a recommended response to the anomaly, or otherwise presents a recommended response and/or associated data, such as visually and/or audibly. In some embodiments, the recommendation generator 256 (e.g., the machine learning model 104) may generate an air quality improvement recommendation that includes an activation of one or more air quality improvement devices, a servicing of a piece of equipment in the domicile, etc. For example, the recommendation generator 256 (e.g., the machine learning model 104) may generate an air quality improvement recommendation that includes a user interface that provides a recommended activation of an air quality improvement device, for example for review (and/or selection) by a user or operator, or otherwise audibly or visually presents the air quality improvement recommendation via a user computing device, such as a device display screen or a voice bot.

Additionally or alternatively, the recommendation generator 256 (e.g., the machine learning model 104) may generate an air quality improvement recommendation that includes information relating to a policy parameter. For example, the recommendation generator 256 (e.g., the machine learning model 104) may generate an air quality improvement recommendation that includes a policy parameter (e.g., discount, amount, coverage, coverage availability, coverage provider, UBI parameters or units, etc.) associated with the air quality improvement recommendation.

In certain embodiments, the recommendation generator 256 may be further configured to communicate the air quality improvement recommendation to one or more devices, systems, and/or environments. For example, the recommendation generator 256 may be configured to communicate the air quality improvement recommendation to the user device 120 (e.g., via the communications interface 202), for example for display (e.g., via the user interface 122) or voice reproduction, such as in the case of a voice bot, chat bot, ChatGPT bot, generative AI bot, etc.

Additionally or alternatively, the recommendation generator 256 may be configured to communicate the air quality improvement recommendation to the database 258 and/or the storage system 160 (e.g., via the communications interface 202 via the network 170), for example for storage and/or subsequent air quality improvement recommendation generation. In some embodiments, the recommendation generator 256 may be configured to communicate the air quality improvement recommendation to the third-party system 130, the provider system 140, and/or the computing system 150 (e.g., via the communications interface 202 via the network 170), for example for storage and/or subsequent analysis (e.g., authorization, verification, etc.).

### Exemplary Air Quality Analysis System & Functionality

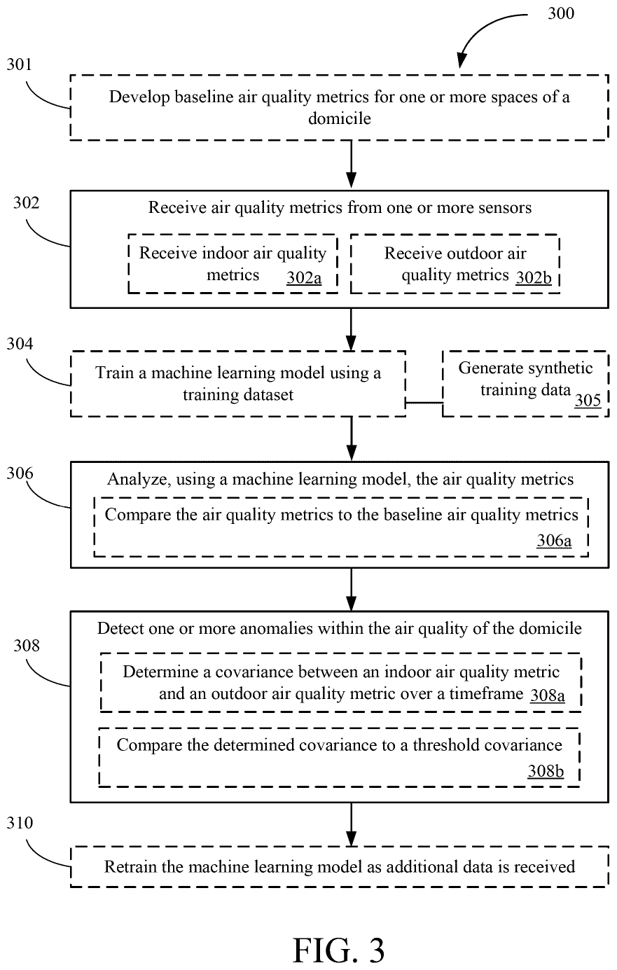

Referring now to FIG. 3, a computer-implemented or computer-based process, shown as process 300, for detecting one or more anomalies within an air quality is shown, according to some embodiments. Computer-implemented process 300 may be implemented by any and/or all the components of the building management system 100 of FIGS. 1-2 (e.g., the air quality analysis system 102, etc.). It should be appreciated that any and/or all the process 300 may be implemented by other systems, devices, and/or components (e.g., components of the building management system 100, the air quality analysis system 102, etc.). Further, it should be appreciated that process 300 may be implemented using additional, different, and/or fewer operations, actions, and/or functionality.

Computer-implemented process 300 may include developing baseline air quality metrics for one or more spaces of a domicile (block 301), according to some embodiments. The baseline air quality metrics may include the threshold amounts for each of the air quality metrics received by the analyzer 252, as described above. In some embodiments, the baseline air quality metrics may be developed based upon one or more health standards (e.g., received from a third-party, such as a health agency), regulatory requirements (e.g., received from a government agency), building standards (e.g., received from a contractor associated with the domicile), policy parameters (e.g., received from the provider system 140), and any other standard/requirement/regulation/guideline related to air quality.

In certain implementations, block 301 may include developing a baseline air quality metric for each of the air quality metrics measurable by the sensor system 110. In some instances, the baseline air quality metrics may be developed according to one or more parameters associated with a particular domicile and/or a space within the domicile (e.g., a bedroom, a family room, a living room, an office space, a basement, a kitchen, a bathroom, etc.). For example, if the domicile includes a single-family home, a baseline air exchange rate may be lower than the baseline air exchange rate in a college dormitory. As another example, the baseline air quality metrics associated with a sensor (e.g., the indoor air quality sensor 112) installed in a bedroom may differ from the baseline air quality metrics associated with a sensor installed in a basement (e.g., a baseline temperature for a basement may be lower than a baseline temperature for a bedroom). As yet another example, if the bedroom is occupied by a baby, the baseline air quality metrics may differ from the baseline air quality metrics associated with a bedroom occupied by an adult. In this way, the baseline air quality metrics developed at block 301 may depend on the domicile in which the air quality analysis system 102 is analyzing an air quality, the space in which a sensor (e.g., a sensor included in the sensor system 110) is installed, and/or an occupant (e.g., resident, user, etc.) of the space in which the sensor is installed. In some embodiments, the baseline air quality metrics may include a threshold air quality index for the domicile.

Computer-implemented process 300 may include receiving air quality metrics from one or more sensors (block 302), according to some embodiments. The one or more sensors may include any of the sensors included in the sensor system 110, as described herein. In some embodiments, the data compiler 250 may receive raw data from the one or more sensors. The data compiler 250 may, from the raw data, identify the one or more air quality metrics (e.g., a particulate level, a carbon dioxide (CO₂) level, an amount of volatile organic compounds (VOCs), a carbon monoxide (CO) level, a humidity percentage, a temperature, a radon level, an amount of formaldehyde, a nitrogen dioxide (NO₂) level, an air exchange rate, etc.). Alternatively, or additionally, the sensor system 110 may be configured to extract the air quality metrics from the raw data.

Block 302 may further include receiving indoor air quality metrics (block 302a) and/or receiving outdoor air quality metrics (block 302b). The indoor air quality metrics may include air quality metrics received from an indoor air quality sensor 112. The outdoor air quality metrics may include air quality metrics received from an outdoor air quality sensor 114. In various embodiments, the data compiler 250 may be configured to distinguish the indoor air quality metrics from the outdoor air quality metrics such that the air quality metrics transmitted to the analyzer 252 may be sorted according to indoor air quality metrics and outdoor air quality metrics.

Computer-implemented process 300 may include training a machine learning model using a training dataset (block 304), according to some embodiments. The machine learning model may include one or more regression trees, deep neural networks, supervised learning models, unsupervised learning models, nearest neighbors, generative adversarial networks (GANs), stable diffusers, generative artificial intelligence (GAI), transformers, or many other types of models. The machine learning model may be trained to analyze air quality metrics and to detect one or more anomalies within the air quality of a domicile based upon the analysis.

Therefore, the training dataset may include training data that is specific to the domicile in which the air quality analysis system 102 is analyzing the air quality (e.g., domicile-specific training data). For example, the training dataset may include historic sensor data from the sensor system 110, a history of an operability of air quality improvement devices within the domicile (e.g., a history of installation, servicing, device failure, etc.). In some instances, the training dataset may include policy parameters associated with a policy related to the domicile and/or a resident of the domicile associated with a policy from the provider system 140.

In some implementations, the computer-implemented process 300 may include generating synthetic training data (block 305), such that the machine learning model may be trained during block 304 using the synthetic training data. For example, the computer-implemented process 300 may include generating synthetic training data when there is insufficient training data associated with the domicile (e.g., insufficient domicile-specific training data). That is, the sensor system 110 may not include historic sensor data, the domicile may not include a history of an operability of air quality improvement devices, and/or the domicile may have recently activated a policy with the provider system 140. In each of these cases, the domicile may not be associated with domicile-specific training data for training the machine learning model, so the machine learning model may be configured to generate synthetic training data based upon domicile-specific training data of other domiciles with similar characteristics (e.g., a similar sensor system 110, similar air quality improvement devices, similar policy parameters, similar residents, etc.) as the domicile having insufficient training data.

Computer-implemented process 300 may include analyzing, using a machine learning model, the air quality metrics (block 306), according to some embodiments. In some embodiments, the analyzer 252 may be configured to analyze the air quality metrics, as described herein. The analyzer 252 may be configured to receive the air quality metrics from the sensor system 110 and/or from the data compiler 250. In certain embodiments, analyzing the air quality metrics may include determining whether the air quality metrics satisfy a threshold amount. In various embodiments, the threshold amount may include the baseline air quality metrics. Analyzing the air quality metrics at block 306 may further include generating an air quality index, as described herein. For example, generating the air quality index may include generating a single score representative of the relative level of air quality in the domicile.

In such embodiments, the block 306 may further include comparing the air quality metrics to the baseline air quality metrics (block 306a). For example, comparing the air quality metrics to the baseline air quality metrics at block 306a may include determining whether the air quality metrics received from the sensor system 110 and/or the data compiler 250 fall below the baseline air quality metrics, satisfy (e.g., meet a threshold amount, fall within a threshold range, etc.) the baseline air quality metrics, or exceed the baseline air quality metrics. In certain embodiments, the baseline air quality metrics may include a threshold air quality index and the comparison at block 306a may including comparing a generated air quality index (e.g., generated by the analyzer 252, as described herein) to the threshold air quality index.

Computer-implemented process 300 may include detecting one or more anomalies within the air quality of the domicile (block 308), according to some embodiments. In some embodiments, the anomaly detector 254 may be configured to detect the one or more anomalies within the air quality of the domicile, as described herein. For example, the one or more anomalies may include at least one of the air quality metrics (e.g., an amount of volatile organic compounds (VOCs), a carbon monoxide (CO) level, a humidity percentage, a temperature, a radon level, an amount of formaldehyde, a nitrogen dioxide (NO₂) level, an air exchange rate, and any other metric associated with an air quality) failing to satisfy (e.g., falling short of, exceeding, etc.) the corresponding baseline air quality metric.

In certain embodiments, the machine learning model may be trained to detect the one or more anomalies. In this way, the machine learning model may be configured to detect the anomalies according to a particular domicile, a space within the domicile, an occupant/user of the space within the domicile, a policy parameter associated with the domicile, etc. For example, as described herein, the baseline air exchange rate for a college dormitory may differ from the baseline air exchange rate for a single-family home. Therefore, the machine learning model may be trained according to data associated with the dormitory (e.g., the domicile-specific training data) for detecting one or more anomalies in the dormitory, while the machine learning model may be trained according to data associated with the single-family home (e.g., the domicile-specific training data) for detecting one or more anomalies in the single-family home. The domicile-specific training data may further include data associated with one or more spaces (e.g., a bedroom, a family room, a living room, an office space, a basement, a kitchen, a bathroom, a patio, etc.) within the domicile (e.g., the dormitory and/or the single-family home), data associated with one or more occupants/users of the spaces within the domicile (e.g., a baby, a child, an adult, an immuno-compromised person, etc.), data relating to a policy associated with the domicile, etc., such that the machine learning model may be configured to detect anomalies within the air quality of the domicile according to the particular domicile.

Block 308 may further include determining a covariance between an indoor air quality metric and an outdoor air quality metric over a timeframe (block 308a). The indoor air quality metric may be received from the indoor air quality sensor 112 and the outdoor air quality metric may be received from the outdoor air quality sensor 114. The indoor air quality metric and the outdoor air quality metric may be received from the sensor system 110 by the data compiler 250 such that the data compiler 250 may distinguish the indoor air quality metric from the outdoor air quality metric. In various embodiments, the analyzer 252 may be configured to determine the covariance for a particular time frame. The covariance may be used to determine whether anomalies within the indoor air quality (e.g., detected by the anomaly detector 254, as described herein) may be attributed to variances in an outdoor air quality.

In certain embodiments, after determining the covariance between the indoor air quality metric and the outdoor air quality metric at block 308a, block 308 may further include comparing the determined covariance to a threshold covariance (block 308b). The threshold covariance may refer to the covariance between the indoor air quality metrics and the outdoor air quality metrics when the indoor air quality metrics satisfy threshold (e.g., baseline, standard, healthy, etc.) air quality metrics. That is, the threshold covariance indicates a natural covariance between an indoor air quality and an outdoor air quality that may be attributable to uncontrollable (e.g., environmental) factors in the outdoor air quality. Therefore, a covariance between the indoor air quality metric and the outdoor air quality metric that differs from the threshold covariance may be attributable to a deficiency within the indoor air quality, rather than to an uncontrollable factor in the outdoor air quality. The analyzer 252 may be configured to compare the determined covariance to the threshold covariance such that the anomaly detector 254 may be configured to detect an anomaly in response to the covariance between the indoor air quality metric and the outdoor air quality metric being less than a threshold covariance.

Computer-implemented process 300 may include retraining the machine learning model as additional data is received (step 310), according to some embodiments. That is, as the air quality analysis system 102 receives additional data (e.g., by performing the functions described herein over time), the machine learning model 104 is retrained based upon the additional data such that the machine learning model 104 may perform more accurate analyses and generate more relevant recommendations for the particular domicile in which the air quality analysis system 102 is analyzing the air quality.

### Air Quality Analysis Recommendation Generation

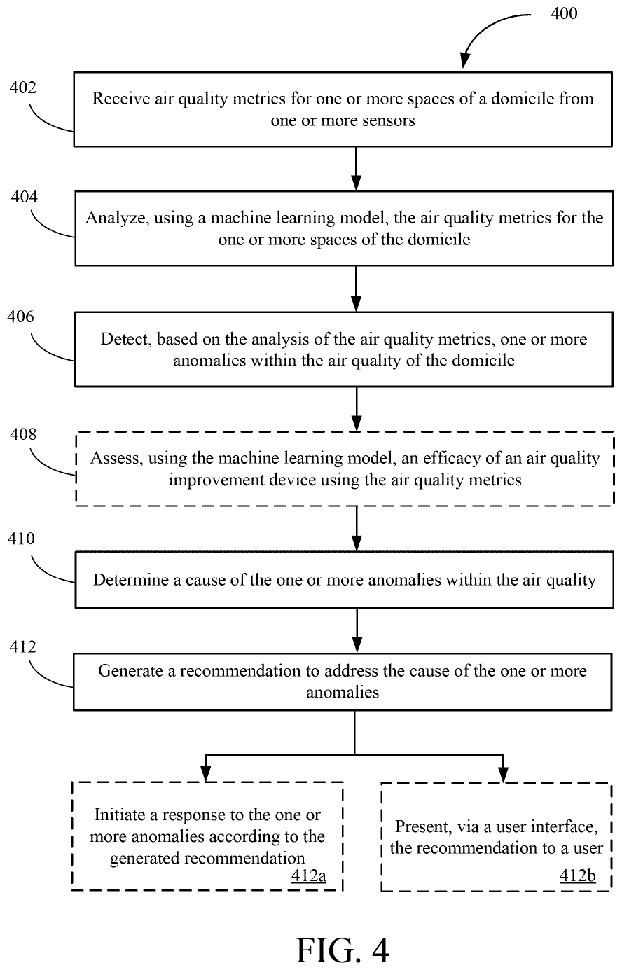

Referring now to FIG. 4, a computer-implemented or computer-based process, shown as process 400 for generating a recommendation to address one or more anomalies within an air quality is shown, according to some embodiments. Process 400 may be implemented by any and/or all the components of the building management system 100 of FIGS. 1-2 (e.g., the air quality analysis system 102, etc.). It should be appreciated that any and/or all the process 400 may be implemented by other systems, devices, and/or components (e.g., components of the building management system 100, the air quality analysis system 102, etc.). It should be appreciated that process 400 may be implemented using additional, different, and/or fewer operations, actions, and/or functionality.

Computer-implemented process 400 may include receiving air quality metrics for one or more spaces of a domicile from one or more sensors (block 402), according to some embodiments. The one or more sensors may include any of the sensors included in the sensor system 110, as described herein. In some embodiments, the data compiler 250 may receive raw data from the one or more sensors. The data compiler 250 may, from the raw data, identify the one or more air quality metrics (e.g., a particulate level, a carbon dioxide (CO₂) level, an amount of volatile organic compounds (VOCs), a carbon monoxide (CO) level, a humidity percentage, a temperature, a radon level, an amount of formaldehyde, a nitrogen dioxide (NO₂) level, an air exchange rate, etc.). Alternatively, or additionally, the sensor system 110 may be configured to extract the air quality metrics from the raw data.

In various embodiments, block 402 may further include receiving indoor air quality metrics and/or receiving outdoor air quality metrics. The indoor air quality metrics may include air quality metrics received from an indoor air quality sensor 112. The outdoor air quality metrics may include air quality metrics received from an outdoor air quality sensor 114. In some embodiments, the data compiler 250 may be configured to distinguish the indoor air quality metrics from the outdoor air quality metrics such that the air quality metrics transmitted to the analyzer 252 may be sorted according to indoor air quality metrics and outdoor air quality metrics.

Computer-implemented process 400 may include analyzing, using a machine learning model, the air quality metrics for the one or more spaces of the domicile (block 404), according to some embodiments. In certain embodiments, the analyzer 252 may be configured to analyze the air quality metrics, as described herein. The analyzer 252 may be configured to receive the air quality metrics from the sensor system 110 and/or from the data compiler 250. According to certain implementations, analyzing the air quality metrics may include generating an air quality index. For example, generating the air quality index may include generating a single score representative of the relative level of air quality in the domicile. In some embodiments, analyzing the air quality metrics may include determining whether the air quality metrics satisfy a threshold amount. For instance, the threshold amount may include baseline air quality metrics (e.g., the baseline air quality metrics developed at block 301 of computer-implemented process 300, as described herein).

In such embodiments, block 404 may further include comparing the air quality metrics to the baseline air quality metrics. For example, comparing the air quality metrics to the baseline air quality metrics may include determining whether the air quality metrics received from the sensor system 110 and/or the data compiler 250 fall below the baseline air quality metrics, satisfy (e.g., meet a threshold amount, fall within a threshold range, etc.) the baseline air quality metrics, or exceed the baseline air quality metrics. In some embodiments, the baseline air quality metrics may include a threshold air quality index and block 404 may include comparing an air quality index (e.g., a single score generated by the analyzer 252, as described herein) to the threshold air quality index.

Computer-implemented process 400 may include detecting, based upon the analysis of the air quality metrics, one or more anomalies within an air quality of the domicile (block 406), according to some embodiments. In some embodiments, the anomaly detector 254 may be configured to detect the one or more anomalies within the air quality of the domicile, as described herein. For example, the one or more anomalies may include at least one of the air quality metrics (e.g., an amount of volatile organic compounds (VOCs), a carbon monoxide (CO) level, a humidity percentage, a temperature, a radon level, an amount of formaldehyde, a nitrogen dioxide (NO₂) level, an air exchange rate, and any other metric associated with an air quality) failing to satisfy (e.g., falling short of, exceeding, etc.) a baseline air quality metric. As another example, the one or more anomalies may be detected based upon an air quality index (e.g., a single score generated by the analyzer 252) failing to meet a threshold air quality index (e.g., a threshold score).

In certain embodiments, a machine learning model may be trained to detect the one or more anomalies. In this way, the machine learning model may be configured to detect the anomalies according to a particular domicile, a space within the domicile, an occupant/user of the space within the domicile, a policy parameter associated with the domicile, etc. For example, as described herein, a baseline air exchange rate for a college dormitory may differ from a baseline air exchange rate for a single-family home. Therefore, the machine learning model may be trained according to data associated with the dormitory (e.g., domicile-specific training data) for detecting one or more anomalies in the dormitory, while the machine learning model may be trained according to data associated with the single-family home (e.g., domicile-specific training data) for detecting one or more anomalies in the single-family home.

The domicile-specific training data may further include data associated with one or more spaces (e.g., a bedroom, a family room, a living room, an office space, a basement, a kitchen, a bathroom, a patio, etc.) within the domicile (e.g., the dormitory and/or the single-family home), data associated with one or more occupants/users of the spaces within the domicile (e.g., a baby, a child, an adult, an immuno-compromised person, etc.), data relating to a policy associated with the domicile, etc., such that the machine learning model may be configured to detect anomalies within the air quality of the domicile according to the particular domicile.

Computer-implemented process 400 may include assessing, using the machine learning model, an efficacy of an air quality improvement device using the air quality metrics (block 408), according to some embodiments. The air quality improvement device may include any device, system, component, etc. configured to control an air quality metric. For example, the air quality improvement device may include an air filtration system and/or an air cleaning system. The air filtration system and/or the air cleaning system may further include one or more components (e.g., a fan, a dehumidifier, a filter, and any other device/component used in an air filtration system and/or an air cleaning system).

The efficacy of the air quality improvement device refers to a performance metric related to the operation of the air quality improvement device. That is, the metric may indicate whether the air quality improvement device is working at full capacity, working at partial capacity, failing to operate, etc. In some embodiments, each air quality improvement device and/or component thereof (e.g., a fan, a dehumidifier, a filter, and any other device/component used in an air filtration system and/or an air cleaning system) may be used to control a particular air quality metric. For example, the dehumidifier may be implemented to control a humidity percentage within a domicile and/or a space of a domicile, while a fan may be implemented to control an air exchange rate within a domicile and/or a space of the domicile. Therefore, the air quality metrics may indicate the efficacy of the air quality improvement device because at full operational capacity, the air quality improvement device may be configured to prevent anomalies within the air quality by controlling the air quality metrics. The one or more anomalies detected based upon the air quality metrics may then reveal that the air quality improvement device used to control the air quality metric is not operating efficiently.

Computer-implemented process 400 may include determining a cause of the one or more anomalies within the air quality (block 410), according to some embodiments. As described herein, the cause of the one or more anomalies may be indicated by the performance of an air quality improvement device that is configured to control air quality metrics. That is, after detecting the one or more anomalies at block 406, the air quality analysis system 102 may be configured to identify an air quality improvement device used to control the air quality metric associated with the anomaly. For example, if the anomaly detector 254 detects an anomaly within a humidity percentage for a space within a domicile, the air quality analysis system 102 may determine the cause of the anomaly to be an inefficient operation of a dehumidifier in the space. In some embodiments, the cause of the one or more anomalies may be determined based upon the efficacy of the air quality improvement device assessed at block 408.

The cause of the one or more anomalies may also be determined by identifying, from a plurality of factors combined to generate an air quality index for the domicile, one or more factors that contribute to the air quality index falling below a threshold air quality index. For example, in a score calculated using a weighted combination of the plurality of factors, there may be a single factor that causes the score to fall below a threshold score (e.g., the threshold air quality index). Therefore, the single factor may be a cause of the one or more anomalies.

Computer-implemented process 400 may include generating a recommendation to address the cause of the one or more anomalies (block 412), according to some embodiments. After determining the cause of the one or more anomalies at block 410, the air quality analysis system 102 may identify an air quality improvement device designed to control the air quality metric associated with the anomaly. Therefore, the recommendation may include an activation, an installation, a servicing, and/or any other action related to the air quality improvement device designed to control the air quality metric associated with the anomaly.

For example, upon determining, at block 410, that the cause of a high humidity percentage may be a malfunctioning of a dehumidifier, the recommendation to address the cause of the anomaly may include scheduling a servicing of the dehumidifier. As another example, the air quality analysis system 102 may determine that a space where the anomaly is detected does not have a dehumidifier installed. In this example, the recommendation to address the cause of the high humidity percentage may include an installation of a dehumidifier.

Block 412 may further include initiating a response to the one or more anomalies according to the generated recommendation (block 412a). That is, block 412a may include automatically performing an action based upon the recommendation generated at block 412. For example, if the anomaly detector 254 determines that an air exchange rate is below a threshold air exchange rate for a particular domicile (e.g., a single-family home), the recommendation generated at block 412 may include activating a fan in the domicile and the response initiated at block 412a may include automatically activating the fan. In certain embodiments, the response may be initiated based on an air quality index (e.g., a single score generated by the analyzer 252, as described herein) falling below a threshold score for an air quality index associated with the domicile.

In various embodiments, block 412 may further include presenting, via a user interface, the recommendation to a user (block 412b). For example, if the anomaly detector 254 determines that a humidity percentage is above a threshold humidity percentage for a space within a domicile (e.g., a bedroom), the recommendation generated at block 412 may include installing a dehumidifier in the space with the high humidity percentage and the recommendation to install the dehumidifier in the space may be presented to a user (e.g., a resident of the domicile, an occupant of the space, etc.) via a user interface (e.g., user interface 122 of the user device 120, computer-generated user interface 500, etc.).

### User Interface Displaying Air Quality Analysis Recommendation

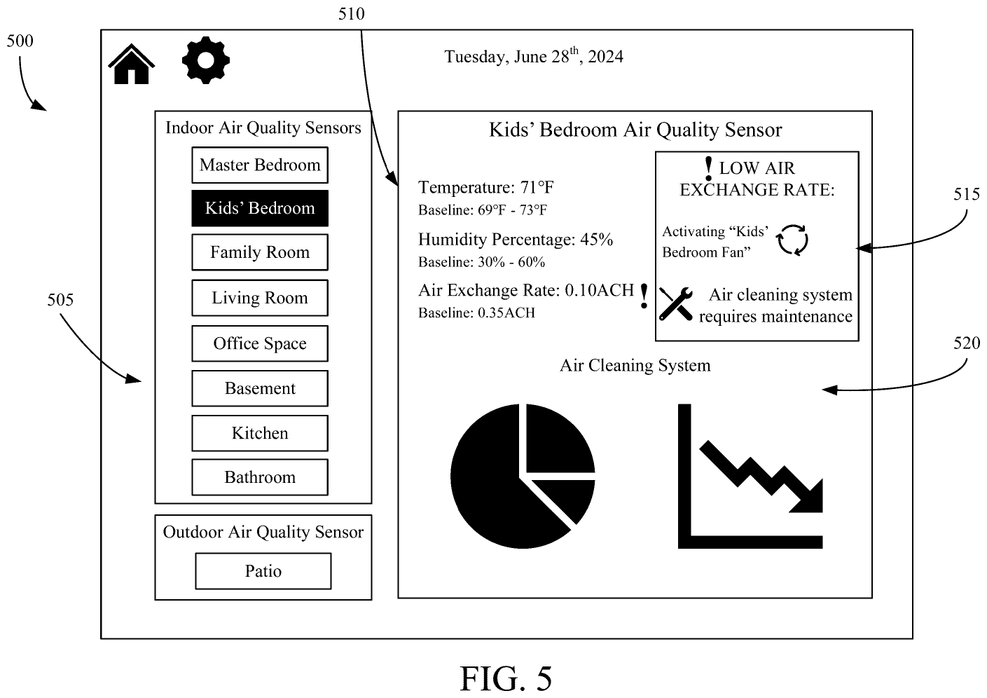

Referring now to FIG. 5, a computer-generated user interface, shown as user interface 500, for displaying a recommended response to one or more anomalies detected within an air quality is shown, according to some embodiments. User interface 500 may be generated by any and/or all the components of the building management system 100 of FIGS. 1-2 (e.g., the air quality analysis system 102, etc.). It should be appreciated that any and/or all the user interface 500 may be generated by other systems, devices, and/or components (e.g., components of the building management system 100, the air quality analysis system 102, etc.). It should be appreciated that in some embodiments, user interface 500 may display additional, different, and/or fewer displays, options, icons, and/or functionality.

Computer-generated user interface 500 may include a list of sensors (item 505), according to some embodiments. The list of sensors may include one or more sensors in the sensor system 110 (e.g., indoor air quality sensor 112, outdoor air quality sensor 114). As shown in FIG. 5, the computer-generated user interface 500 may distinguish the indoor air quality sensor(s) from the outdoor air quality sensor(s). For example, the indoor air quality sensors may be installed in an interior space of a domicile.

As shown in FIG. 5, the indoor air quality sensors may include sensors in a master bedroom, a kids' bedroom, a family room, a living room, an office space, a basement, a kitchen, a bathroom, and/or any other space interior to the domicile. The outdoor air quality sensor may include a patio and/or any other space exterior to the domicile.

In various embodiments, each of the sensors included in the list of sensors may be represented by a selectable element (e.g., a hyperlinked text, a button, a toggle, etc.), such that a user (e.g., a resident of the domicile, a user of the user device 120, a policyholder of a policy with the provider system 140, etc.) may receive, via the computer-generated user interface 500, information associated with the corresponding sensor upon engaging with (e.g., selecting, clicking on, and/or otherwise interacting with) a selectable element among the list of sensors. For example, FIG. 5 shows that an indoor air quality sensor in a kids' bedroom has been selected (e.g., depicted by the contrasting color scheme of the text “Kids' Bedroom” as compared to the text representing each of the other sensors in the list of sensors).

Computer-generated user interface 500 may include one or more air quality metrics (item 510), according to some embodiments. The one or more air quality metrics may be received from a sensor in the list of sensors (e.g., item 505). In some embodiments, the one or more air quality metrics may be presented via the computer-generated user interface 500 after the user engages with a selectable element representing a sensor in the list of sensors, as described herein. For example, after the user selects the “Kids' Bedroom” indoor air quality sensor from the list of sensors, the computer-generated user interface 500 may display the air quality metrics measured by the indoor air quality sensor located in the kids' bedroom (e.g., the space) of the domicile. For example, the air quality metrics may include a temperature, a humidity percentage, an air exchange rate, and/or any additional data/metric measured by the selected sensor.

In various embodiments, the air quality metrics may also include an air quality index (e.g., a score representative of the relative level of air quality in a space associated with the selected sensor). For example, the air quality index may include a single score generated using a weighted combination of a plurality of factors (e.g., generated by the analyzer 252, as described herein). In this example, the computer-generated user interface 500 may include the single score alone and/or in combination with information related to the component factors that are used in the generation of the score. In some embodiments, the information related to the component factors may further include one or more reasons for the single score being high and/or low, as compared to a threshold score.

As shown in FIG. 5, the air quality metrics may include a current measurement from the selected sensor and a baseline measurement (e.g., the baseline air quality metric developed during block 301 of computer-implemented process 300). In some embodiments, the baseline measurement may include a range and/or a threshold value for the air quality metric.

As described herein, the baseline measurements may be specific to a space within the domicile. Specifically, the baseline measurements displayed on the computer-generated user interface 500 may correspond to the domicile, the space within the domicile, one or more occupants of the space, a policy parameter, etc., associated with the selected sensor from the list of sensors (e.g., item 505). For example, as shown in FIG. 5, if a user selects the kids' bedroom indoor air quality sensor, the baseline measurements for the air quality metrics displayed on the computer-generated user interface 500 may include baseline measurements according to the domicile (e.g., a single-family home), the space (e.g., a bedroom), the occupants of the space (e.g., kids), etc.

In this example, the temperature measured by the indoor air quality sensor may be 71° F., the humidity percentage measured by the indoor air quality sensor may be 45%, and the air exchange rate measured by the indoor air quality sensor may be 0.10 ACH. Continuing with the same example and as shown in FIG. 5, the baseline temperature for the kids' bedroom may include a range of 69° F.-73° F., the baseline humidity percentage for the kids' bedroom may include a range of 30%-60%, and the baseline air exchange rate for the kids' bedroom may include a value of 0.35 ACH.

As shown in FIG. 5, the computer-generated user interface 500 may further indicate (e.g., by the exclamation point icon and/or any other indication) an air quality metric that fails to satisfy the corresponding baseline measurement. In this example, the air exchange rate measured by the indoor air quality sensor located in the kids' bedroom (e.g., 0.10 ACH) fails to satisfy the baseline measurement (e.g., 0.35 ACH) for the air exchange rate in the kids' bedroom. As another example, the computer-generated user interface 500 may indicate one or more factors used within an air quality index calculation that cause the air quality index to fall below a threshold air quality index.

Computer-generated user interface 500 may include a recommendation in response to an anomaly detected within an air quality of a domicile (item 515), according to some embodiments. In some embodiments, item 515 may include a plurality of responses to the anomaly detected within the air quality of the domicile. For example, the recommendation may include a response that may be automatically initiated by the air quality analysis system 102 (e.g., the response initiated at block 412a of the computer-implemented process 400). Additionally or alternatively, the recommendation may include a suggestion to a user of the computer-generated user interface 500 (e.g., the recommendation presented at block 412b of the computer-implemented process 400). In certain embodiments, the recommendation may relate to an air quality improvement device that is used to control the air quality metric corresponding to the anomaly (e.g., the air quality improvement device identified as a cause of the anomaly, as described with reference to computer-implemented process 400).

As shown in FIG. 5, the recommendation included on the computer-generated user interface 500 may relate to an anomaly detected within the air exchange rate in the kids' bedroom of the domicile. In this example, the recommendation may include an automatic activation of an air quality improvement device (e.g., a fan) in the kids' bedroom. As shown in FIG. 5, the recommendation may further include an indication that an air quality improvement device (e.g., an air cleaning system) requires maintenance. In this example, the fan and the air cleaning system may be used to control the air exchange rate within a space of the domicile.

In some embodiments, the recommendation may further include one or more selectable elements that allow a user of the computer-generated user interface 500 to take action in response to receiving the recommendation. For example, an icon (e.g., a circulation icon, as shown in FIG. 5) and/or a hyperlinked text (e.g., Activating “Kids' Bedroom Fan”) may be a selectable element configured to allow the user to perform an action relating to the activation of the fan. After engaging with the icon and/or the hyperlinked text, the user may receive an option to pause activation of the fan, adjust a speed of the fan, activate additional air quality improvement devices, and/or perform any other function related to the air quality within the kids' bedroom.

Alternatively or additionally, an icon (e.g., a wrench icon, as shown in FIG. 5) and/or a hyperlinked text (e.g., “air cleaning system requires maintenance”) may be a selectable element configured to allow the user to perform an action relating to the maintenance required for the air cleaning system. For example, after engaging with the icon and/or the hyperlinked text, the user may receive an option to schedule maintenance for the air cleaning system, purchase a component of the air cleaning system that requires a replacement, and/or view additional data/information related to the maintenance required for the air cleaning system.

Computer-generated user interface 500 may include performance metrics associated with an air quality improvement device (item 520), according to some embodiments. In various embodiments, the performance metrics may include the efficacy of the air quality improvement device assessed at block 408 of computer-implemented process 400. The air quality improvement device may be an air quality improvement device installed with a domicile and/or a space within the domicile in which the selected sensor from the list of sensors included in the computer-generated user interface 500 is installed. For example, if the selected sensor is an indoor air quality sensor in a kids' bedroom, the performance metrics may relate to an air quality improvement device within the kids' bedroom and/or otherwise inside the domicile.

More specifically, the performance metrics may relate to an air quality improvement device identified as a cause (e.g., the cause determined at block 410 of computer-implemented process 400, according to some embodiments) of one or more anomalies detected within the air quality metrics. Continuing with the same example, and as shown in FIG. 5, if the air quality metrics from the indoor air quality sensor in the kids' bedroom indicate an anomaly within the air exchange rate in the kids' bedroom, the performance metrics included on the computer-generated user interface 500 may relate to an air cleaning system used to maintain/control an air exchange rate in the kids' bedroom and/or in the domicile as a whole.

### Exemplary Machine Learning and Generative AI

As discussed elsewhere, some embodiments may utilize machine learning, generative artificial intelligence, or other advanced computing techniques. As such, generative artificial intelligence (AI) models (also referred to as generative machine learning (ML) models) and/or other AI/ML models discussed herein may be implemented via and/or coupled to one or more voice bots and/or chatbots that may be configured to utilize artificial intelligence and/or machine learning techniques. For instance, the voice and/or chatbot may be a ChatGPT chatbot and/or a ChatGPT-based bot. The voice and/or chatbot may employ supervised, unsupervised, and/or semi-supervised machine learning techniques, which may be followed by, and/or used in conjunction with, reinforced and/or reinforcement learning techniques. The voice bot, chatbot, ChatGPT bot, ChatGPT-based bot, and/or other such generative model may generate audible or verbal output, text or textual output, visual or graphical output, output for use with speakers and/or display screens of a mobile computing device, and/or other types of output for user and/or other computer or bot consumption.

Noted above, in some embodiments, a chatbot or other computing device may be configured to implement machine learning, such that the computing device “learns” to analyze, organize, and/or process data without being explicitly programmed. Machine learning and/or artificial intelligence may be implemented through machine learning methods and algorithms. In one exemplary embodiment, a machine learning module may be configured to implement the ML methods and algorithms.

As used herein, a voice bot, chatbot, ChatGPT bot, ChatGPT-based bot, and/or other such generative model (referred to broadly as “chatbot” herein) may refer to a specialized system for implementing, training, utilizing, and/or otherwise providing an AI or ML model to a user for dialogue interaction (e.g., “chatting”). Depending on the embodiment, the chatbot may utilize and/or be trained according to language models, such as natural language processing (NLP) models and/or large language models (LLMs). Similarly, the chatbot may utilize and/or be trained according to generative adversarial network (GAN) techniques, such as the machine learning techniques, algorithms, and systems described in more detail below. In some implementations, a multimodal model, such as a multimodal generative pretrained transformer or other multimodal transformer model, may be utilized.

The chatbot may receive inputs from a user via text input, spoken input, gesture input, etc. The chatbot may then use AI and/or ML techniques as described herein to process and analyze the input before determining an output and displaying the output to the user. Depending on the embodiment, the output may be in a same or different form than the input (e.g., spoken, text, gestures, etc.), may include images, and/or may otherwise communicate the output to the user in an overarching dialogue format.

In various embodiments, at least one of a plurality of ML methods and algorithms may be applied to implement and/or train the chatbot, which may include but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, a chatbot ML module employs supervised learning, which involves identifying patterns in existing data to make predictions about subsequently received data. Specifically, the chatbot ML module may be “trained” using training data, which includes example inputs and associated example outputs. Based upon the training data, the chatbot ML module may generate a predictive function which maps outputs to inputs and may utilize the predictive function to generate ML outputs based upon data inputs. The exemplary inputs and exemplary outputs of the training data may include any of the data inputs or ML outputs described above. In the exemplary embodiment, a processing element may be trained by providing it with a large sample of data with known characteristics or features.

In another embodiment, the chatbot ML module may employ unsupervised learning, which involves finding meaningful relationships in unorganized data. Unlike supervised learning, unsupervised learning does not involve user-initiated training based upon example inputs with associated outputs. Rather, in unsupervised learning, the chatbot ML module may organize unlabeled data according to a relationship determined by at least one ML method/algorithm employed by the chatbot ML module. Unorganized data may include any combination of data inputs and/or ML outputs as described above.

In yet another embodiment, the chatbot ML module may employ semi-supervised learning, which involves using thousands of individual supervised machine learning iterations to generate a structure across the multiple inputs and outputs. In this way, the chatbot ML module may be able to find meaningful relationships in the data, similar to unsupervised learning, while leveraging known characteristics or features in the data to make predictions via a ML output.

In yet another embodiment, the chatbot ML module may employ reinforcement learning, which involves optimizing outputs based upon feedback from a reward signal. Specifically, the chatbot ML module may receive a user-defined reward signal definition, receive a data input, utilize a decision-making model to generate a ML output based upon the data input, receive a reward signal based upon the reward signal definition and the ML output, and alter the decision-making model so as to receive a stronger reward signal for subsequently generated ML outputs. Other types of machine learning may also be employed, including deep or combined learning techniques.

In certain embodiments, the chatbot ML module may be used in conjunction with the machine vision, image recognition, object identification, AR glasses, VR headsets, other input/output devices, and/or other image processing techniques discussed below. Additionally or alternatively, in some embodiments, the chatbot ML module may be configured and/or trained to implement one or more aspects of the machine vision, image recognition, objection identification, and/or other image processing techniques discussed below.

### Additional Considerations

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied, or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor and may be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for storage of a computer program.

In some embodiments, a computer program is provided, and the program is embodied on a computer readable medium. In some embodiments, the system is executed on a single computer system, without requiring a connection to a server computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process may be practiced independent and separate from other components and processes described herein. Each component and process may also be used in combination with other assembly packages and processes.

The construction and arrangement of the systems and methods as shown in the various example embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method operations, actions, or functionality may be varied or re-sequenced according to alternative embodiments. Other substitutions, modifications, changes, and omissions may be made in the design, operating conditions, and arrangement of the example embodiments without departing from the scope of the present disclosure.

As used herein, an element or operation recited in the singular and proceeded with the word “a” or “an” should be understood as not excluding plural elements or operations, unless such exclusion is explicitly recited. Furthermore, references to “exemplary embodiment,” “one embodiment,” or “some embodiment” of the present disclosure are not intended to be interpreted as excluding the existence of additional embodiments that also incorporate the recited features.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The patent claims at the end of this document are not intended to be construed under 35 U.S.C. § 112(f) unless traditional means-plus-function language is expressly recited, such as “means for” or “step for” language being expressly recited in the claim(s).

Although the Figures show a specific order of method operations, actions, or functionality, the order of such may differ from what is depicted. Also, two or more operations, actions, or functionalities may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection operations or actions, processing operations or actions, comparison operations or actions, and decision operations or actions.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The term “coupled” and variations thereof, as used herein, means the joining of two members directly or indirectly to one another. Such joining may be stationary (e.g., permanent, or fixed) or moveable (e.g., removable, or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

In various implementations, the functionality and operations described herein may be performed on one processor or in a combination of two or more processors. For example, in some implementations, the various operations could be performed in a central server or set of central servers configured to receive data from one or more devices (e.g., edge computing devices/controllers) and perform the operations. In some implementations, the operations may be performed by one or more local controllers or computing devices (e.g., edge devices), such as controllers dedicated to and/or located within a particular industrial environment or portion of an industrial environment. Additionally or alternatively, the operations may be performed by a combination of one or more central or offsite computing devices/servers and one or more local controllers/computing devices. All such implementations are contemplated within the scope of the present disclosure.

Further, unless otherwise indicated, when the present disclosure refers to one or more computer-readable storage media and/or one or more controllers, such computer-readable storage media and/or one or more controllers may be implemented as one or more central servers, one or more local controllers or computing devices (e.g., edge devices), any combination thereof, or any other combination of storage media and/or controllers regardless of the location of such devices.

What is claimed is:

1. A system for analyzing an air quality of a domicile, the system comprising:
one or more memory devices having instructions stored thereon that, when executed by one or more processors, cause the one or more processors to perform operations comprising:
receiving air quality metrics for one or more spaces of the domicile from one or more sensors;
analyzing, using a machine learning model, the air quality metrics for the one or more spaces of the domicile; and
detecting, based upon the analysis of the air quality metrics using the machine learning model, one or more anomalies within the air quality of the domicile.

2. The system of claim 1, wherein the one or more anomalies comprise a monitored level of at least one of volatile organic compounds (VOCs), carbon monoxide (CO), humidity, temperature, radon, formaldehyde, nitrogen dioxide (NO₂), or an air exchange rate exceeding a threshold level.

3. The system of claim 1, wherein the operations further comprise assessing, using the machine learning model, an efficacy of an air quality improvement device using the air quality metrics, the air quality improvement device configured to improve the air quality of the domicile.

4. The system of claim 3, wherein the air quality improvement device comprises at least one of an air filtration system or an air cleaning system.

5. The system of claim 1, wherein the operations further comprise developing baseline air quality metrics for the domicile, and wherein the one or more anomalies are detected based upon the baseline air quality metrics for the domicile.

6. The system of claim 1, wherein the operations further comprise:
predicting, by the machine learning model, a cause of the one or more anomalies;
generating, by the machine learning model, a recommendation to address the cause of the one or more anomalies; and
presenting, via a user interface, the recommendation to a user.

7. The system of claim 6, wherein the cause of the one or more anomalies may be determined based upon a cross-correlation between the air quality metrics and data from one or more devices in the domicile.

8. The system of claim 6, wherein the recommendation comprises at least one of servicing a piece of equipment of the domicile or activating an air quality improvement device.

9. The system of claim 8, wherein the operations further comprise automatically initiating the at least one of the servicing the piece of equipment of the domicile or activating the air quality improvement device in response to detecting the one or more anomalies.

10. The system of claim 1, wherein the one or more sensors comprise a plurality of sensors configured to be installed within a plurality of spaces of the domicile, the plurality of spaces comprising two or more of a bedroom, a family room, a living room, an office space, a kitchen, a bathroom, or a basement.

11. The system of claim 10, wherein:
receiving the air quality metrics comprises receiving the air quality metrics for the plurality of spaces;
analyzing the air quality metrics comprises analyzing, using the machine learning model, the air quality metrics for the plurality of spaces;
detecting the one or more anomalies comprises detecting a first anomaly within the air quality of a first space of the plurality of spaces of the domicile; and
the operations further comprise providing an indication of the first anomaly and the first space of the domicile for which the anomaly is detected.

12. The system of claim 1, wherein the one or more sensors comprise one or more indoor air quality sensors of the domicile and the air quality metrics comprise indoor air quality metrics, and wherein the operations further comprise:
receiving outdoor air quality metrics from at least one of one or more outdoor air quality sensors coupled to or proximate to an exterior of the domicile or an external system configured to provide outdoor air quality data for a geographic location of the domicile; and
analyzing, using the machine learning model, the outdoor air quality metrics;
wherein detecting the one or more anomalies comprises detecting a first anomaly of the one or more anomalies by the machine learning model using both the indoor air quality metrics and the outdoor air quality metrics.

13. The system of claim 12, wherein detecting the first anomaly comprises:
determining, by the machine learning model, a covariance between a first indoor air quality metric of the indoor air quality metrics and a first outdoor air quality metric of the outdoor air quality metrics over a timeframe; and
detecting the first anomaly responsive to the covariance of the first indoor air quality metric and the first outdoor air quality metric being less than a threshold covariance.

14. The system of claim 1, wherein the operations further comprise generating an indoor air quality index for at least one of the domicile or the one or more spaces of the domicile based upon a combination of the air quality metrics.

15. A computer-implemented method for analyzing an air quality of a domicile, the computer-implemented method comprising:
receiving, using one or more processors and one or more computer-readable storage media having instructions stored thereon executable by the one or more processors, air quality metrics for one or more spaces of the domicile from one or more sensors;
training, using the one or more processors, a machine learning model;
analyzing, using the one or more processors and using the machine learning model, the air quality metrics for the one or more spaces of the domicile;
detecting, using the one or more processors and based upon the analysis of the air quality metrics using the machine learning model, one or more anomalies within the air quality of the domicile;
predicting, using the one or more processors and using the machine learning model, a cause of the one or more anomalies, wherein the cause may be predicted using data from at least one of the one or more sensors or one or more other devices in the domicile;
generating, using the one or more processors and using the machine learning model, a recommendation to address the cause of the one or more anomalies; and
automatically initiating, using the one or more processors, an action responsive to the recommendation.

16. The computer-implemented method of claim 15, wherein training the machine learning model comprises training the machine learning model using domicile-specific training data, the domicile-specific training data comprising training data specific to the domicile for which the computer-implemented method is analyzing the air quality.

17. The computer-implemented method of claim 16, further comprising generating, using the one or more processors and using the machine learning model, a synthetic training dataset, wherein the machine learning model is trained using the synthetic training dataset until the domicile-specific training data reaches a threshold amount.

18. The computer-implemented method of claim 15, wherein the one or more sensors comprise one or more indoor air quality sensors of the domicile and the air quality metrics comprise indoor air quality metrics, the computer-implemented method further comprising:
receiving, using the one or more processors, outdoor air quality metrics from at least one of one or more outdoor air quality sensors coupled to or proximate to an exterior of the domicile or an external system configured to provide outdoor air quality data for a geographic location of the domicile;
determining, using the one or more processors and using the machine learning model, a covariance between a first indoor air quality metric of the indoor air quality metrics and a first outdoor air quality metric of the outdoor air quality metrics over a timeframe; and
detecting, using the one or more processors, the first anomaly responsive to the covariance of the first indoor air quality metric and the first outdoor air quality metric being less than a threshold covariance.

19. A non-transitory computer readable medium comprising instructions stored thereon that, when executed by one or more processors, cause the one or more processors to perform operations comprising:
developing, using a machine learning model, baseline air quality metrics for a plurality of spaces of a domicile;
receiving air quality metrics for the plurality of spaces of the domicile from a plurality of sensors configured to be installed within the plurality of spaces of the domicile;
analyzing, using the machine learning model, the air quality metrics for the plurality of spaces of the domicile based upon the baseline air quality metrics for the plurality of spaces of the domicile;
detecting, based upon the analysis of the air quality metrics using the machine learning model, a first anomaly within an air quality of a first space of the plurality of spaces of the domicile;
assessing, using the machine learning model, an efficacy of an air quality improvement device using the air quality metrics, the air quality improvement device configured to improve the air quality of the first space of the plurality of spaces of the domicile;
determining, based upon the efficacy of the air quality improvement device, a cause of the detected first anomaly; and
initiating a response to the first detected anomaly, wherein the response to the first detected anomaly comprises a servicing of the air quality improvement device.

20. The non-transitory computer readable medium of claim 19, wherein the air quality metrics comprise at least one of a particulate level or a carbon dioxide (CO₂) level.
